(19) United States
(12) Patent Application Publication
(10) Pub. No.: US 2021/0146365 A1
(43) Pub. Date: May 20, 2021

(54) Techniques for high-throughput fluid exchange in droplets

(71) Applicant: NEW YORK GENOME CENTER, INC., NEW YORK, NY (US)

(72) Inventor: William Stephenson, Brooklyn, NY (US)

(21) Appl. No.: 16/633,196

(22) PCT Filed: Jul. 24, 2018
(86) PCT No.: PCT/US18/43365

§ 371 Date: Jan. 23, 2020

Related U.S. Application Data
(60) Provisional application No. 62/536,076, filed on Jul. 24, 2017.

(51) Int. Cl. B01L 3/00; G01N 15/14; G01N 35/08

(52) U.S. Cl. CPC B01L 3/502784; B01L 3/50273; G01N 15/1475; G01N 35/085; G01N 2015/1497; B01L 2300/0864; B01L 2400/043; G01N 2015/149; B01L 2200/0673

(57) ABSTRACT

Techniques include a substrate having a microchannel, first and second microchannel branches, and a fork joining the microchannel upstream and the branches downstream. The microchannel passes a continuous stream of droplets, having a first fluid with magnetic particles, separated by a spacer fluid. A picoinjector, disposed along the microchannel, includes both: a supply channel connected to the microchannel by an aperture on a first side of the microchannel; and, a pair of electrodes on an opposite side. The picoinjector injects a volume of a second fluid into a first droplet when the pair of electrodes carries a certain voltage difference. A first magnet introduces a magnetic field into the microchannel between the picoinjector and the fork to move magnetic particles in the first droplet toward the first side of the microchannel before the droplet is split at the fork to produce output droplets of the second fluid with magnetic particles.

### CROSS REFERENCE TO RELATED APPLICATIONS

This application claims the benefit of U.S. Provisional Application No. 62/536,076, filed Jul. 24, 2017, which is hereby incorporated by reference in its entirety.

### BACKGROUND

Microfluidic devices are used extensively for the capture, detection, classification, or quantification of molecules, molecular complexes, viruses, cells and particulates in environmental or biological samples. These devices include one or more microchannels of sub-millimeter (mm, 1 mm=10⁻³ meters) cross section formed in a chip of an inert material, which direct flow of one or more fluids from one or more corresponding reservoirs to interact with each other in a reaction chamber or at a detector or both.

Microfluidics technology has recently emerged as a powerful means to manipulate fluids at a microscale and fully integrate many components and steps for complex yet very precise biochemical analyses. Some of the promising applications include the development of inexpensive diagnostic devices that can be deployed in low-resource settings especially to meet global health challenges such as the lack of facilities and personnel to carry out medical diagnostics. Current microfluidic devices fall into either droplet-based (multiphase) or continuous-flow (single phase) systems. To successfully manipulate these fluids, a number of control strategies have been proposed which often require use of pumps and valves, either integrated on chip or off-chip. A majority of commercially available systems depend actively on external pumps, vacuum and pressure sources, or depend passively on capillary filling. Even for integrated on-chip systems, the valves are controlled by external macro-scale elements, such as computers, and require power sources.

Exchanging buffers/fluids (also called “washing” herein) in molecular biology is a common process that involves replacing one solution with another solution of different composition, typically using magnetic beads as a solid support to retain species of interest (such as cells, organelles, proteins, nucleic acids, and other molecules, collectively referenced herein as a “target”) to be transferred to the new buffer. This is difficult to perform in droplets due to their non-stationary nature (compared to a macroscale well or vial). Droplet microfluidic approaches to washing have been more difficult or lower throughput than other droplet microfluidic unit operations such as sorting or injection.

### SUMMARY

Techniques are provided for high-throughput exchange of fluids in droplets or washing of magnetic particles with or without attached targets in droplets. In various embodiments, a picoinjector and a magnet and a fork in a microchannel are combined to operate together. The picoinjector adds a second fluid to a first fluid in a droplet. The magnet relocates magnetic particles, such as paramagnetic beads, from a portion of the droplet dominated by the first fluid into a portion of the droplet dominated by the injected second fluid. The fork separates the portion with the second fluid and relocated particles from a portion of the droplet with the first fluid and any residual particles. In washing embodiments, the droplet with the second fluid and relocated particles are collected or used. In removal embodiments, the droplet with the first fluid and few, if any, residual particles is collected and used. In some removal embodiments, the first fluid and the second fluid are the same. In some embodiments, both droplets are collected or used.

As used herein, a microchannel is a channel with width and depth each in a range from 1 to 1000 microns (1 micron, also called a micrometer, μm, =10⁻⁶ meters) and length longer than both width and depth. As used herein, a microparticle is a particle with width, depth and length each in the range from 0.5 to 1000 microns; a nanoparticle is a particle with width depth and length each in the range from 1 to 500 nanometers (nm, 1 nm=10⁹ meters) and a “particle” refers to either or both a microparticle and a nanoparticle. As used herein, a magnetic particle is a particle that includes a material that is permanently magnetized, or paramagnetic, i.e., becomes magnetized in the presence of a magnetic field and move toward the applied field, including superparamagnetic, or diamagnetic i.e., becomes magnetized in the presence of a magnetic field and moves away from the applied field. As used herein, a picoinjector is a device configured to inject a volume of a fluid in which the volume is in a range from 1 to 1000 picoliters (pL, 1 pL=10⁻¹² liters). As used herein, a fluid is a liquid or a gas. As used herein, a droplet is a configuration of a fluid of millimeter (mm, 1 mm=10⁻³ meters) to micron dimensions, including any material or particles dispersed or aggregated therein, bounded by a different fluid called a spacer fluid, including any material or particles dispersed or aggregated therein. In many cases, the fluid droplet is hydrophobic or hydrophilic and the spacer fluid is the opposite, i.e., hydrophilic or hydrophobic, respectively, making the droplet and spacer fluid immiscible.

In a first set of embodiments, an apparatus includes a substrate having formed thereon a microchannel, a plurality of microchannel branches comprising a first microchannel branch and a different second microchannel branch, and a fork comprising a junction between the microchannel upstream of the fork and the plurality of microchannel branches downstream of the fork. The microchannel is configured to pass a continuous stream of a plurality of droplets comprising a first fluid with a plurality of magnetic particles. The plurality of droplets is separated in the stream by a spacer fluid. The apparatus also includes a picoinjector disposed along the microchannel. The picoinjector includes: a supply channel formed in the substrate and connected to the microchannel by an aperture on a first side of the microchannel; and, a pair of electrodes on an opposite side of the microchannel. The aperture is a distance D upstream of the fork. The picoinjector is configured to inject through the aperture a volume of a second fluid into a first droplet of the plurality of droplets in the stream when the pair of electrodes carries at least a certain voltage difference. The apparatus also includes a first magnet disposed adjacent to the microchannel between the picoinjector and the fork. The first magnet is configured to introduce a magnetic field into the microchannel between the picoinjector and the fork to move magnetic particles in the first droplet toward the first side of the microchannel.

In some embodiments of the first set, a different second magnet is disposed adjacent to the microchannel upstream of the picoinjector and configured to introduce a magnetic field into the microchannel upstream of the aperture of the picoinjector to move magnetic particles in the first droplet toward the first side of the microchannel.

In some embodiments of the first set, the first microchannel branch is spaced relative to the second microchannel branch in a direction parallel to a direction in which the magnetic particles are forced by the field of the first magnet. In these embodiments, a cross sectional area of the first microchannel branch is R1 times a cross sectional area of the second microchannel branch. In these embodiments, the picoinjector is configured to inject a volume that is R2 times a volume of the first droplet upstream of the aperture, where R1≈R2. In some of these embodiments, R1 is different from 1, e.g., R1>1, or R1<1.

In some embodiments of the first set, a distance D from the aperture to the fork divided by a migration time (T_{M}) defines a speed of the stream during operation. In these embodiments, T_{M} is short compared to a time for the first fluid to mix with the second fluid in the droplet and long compared to a time for the magnetic particles to move in the magnetic field of the first magnet at least a tenth of the width of the microchannel between the electrodes and the fork. In some embodiments of the first set, D is in a range from 10 microns to 1000 microns, e.g., is 330 microns.

In some embodiments of the first set, the microchannel has a first cross sectional area in the vicinity of the aperture that is less than a second cross sectional area of the microchannel between the electrodes and the fork. In some embodiments of the first set, the microchannel has a first cross sectional area in the vicinity of the aperture that is less than a second cross sectional area of the microchannel upstream of the aperture.

In some embodiments of the first set, the microchannel has a first cross sectional area immediately upstream of the aperture that is less than a second cross sectional area of the microchannel immediately downstream of the aperture. In some of these embodiments, the second cross sectional area is (1+R3) times the first cross sectional area; the picoinjector is configured to inject a volume that is R2 times a volume of the first droplet upstream of the aperture; and, R2 is based on R3.

In some embodiments of the first set, the first magnet is an electromagnet. In other embodiments of the first set, the first magnet is a permanent magnet, such as a rare earth Neodymium magnet.

In a second set of embodiments, a method includes causing a stream of a plurality of droplets separated by a spacer fluid to flow through a device, wherein each droplet of the plurality of droplets comprises a first fluid with a plurality of magnetic particles. The device includes a microchannel, a plurality of microchannel branches including a first microchannel branch and a different second microchannel branch, a fork comprising a junction between the microchannel upstream of the fork and the plurality of microchannel branches downstream of the fork, a picoinjector disposed along the microchannel, and a first magnet. The picoinjector includes a supply channel connected to the microchannel by an aperture on a first side of the microchannel and a pair of electrodes on an opposite side of the microchannel. The aperture is a distance D upstream of the fork. The first magnet is disposed adjacent to the microchannel between the picoinjector and the fork. The method also includes supplying a second fluid to the supply channel Still further, the method includes applying a voltage difference to the pair of electrodes when a first droplet of the plurality of droplets is in contact with the second fluid at the aperture to inject through the aperture a volume of the second fluid into the first droplet. Yet further the method includes introducing from the first magnet a magnetic field into the microchannel between the picoinjector and the fork to move magnetic particles in the first droplet toward the first side of the microchannel. Still further, the method includes collecting an output droplet from the first microchannel branch.

In some embodiments of the second set, the first microchannel branch is spaced relative to the second microchannel branch in a direction parallel to a direction in which the magnetic particles are forced by the field of the first magnet. The output droplet is divided from the first droplet at the fork. As a result, the output droplet is made up of the second fluid and at least some of the plurality of magnetic particles.

In some embodiments of the second set, each magnetic particle of the plurality of magnetic particles is a paramagnetic particle or a superparamagnetic particle. In some embodiments of the second set, each magnetic particle is connected to a species of interest to be washed with the second fluid.

In some embodiments of the second set, the output droplet comprises the second fluid and most of the plurality of magnetic particles. In some of these embodiments, the output droplet comprises more of the second fluid than the first fluid, whereby the magnetic particles have been washed by the second fluid.

In a third set of embodiments, a system includes the above apparatus, one or more pressure actuators, one or more processors and one or more computer-readable media including one or more sequences of instructions. The computer-readable medium and the one or more sequences of instructions are configured to, with the processor, cause the system perform at least the following. The system operates a pressure actuator to cause a stream of a plurality of droplets separated by a spacer fluid to flow through the apparatus, wherein each droplet of the plurality of droplets comprises a first fluid with a plurality of magnetic particles. The system operates a pressure actuator to supply a second fluid to the supply channel. The system also applies a voltage difference to the pair of electrodes when a first droplet of the plurality of droplets is in contact with the second fluid at the aperture to inject through the aperture a volume of the second fluid into the first droplet. In addition, the system introduces from the first magnet a magnetic field into the microchannel between the picoinjector and the fork to move magnetic particles in the first droplet toward the first side of the microchannel. Even further, the system collects an output droplet from the first microchannel branch.

In some embodiments of the third set, the first microchannel branch is spaced relative to the second microchannel branch in a direction parallel to a direction in which the magnetic particles are forced by the field of the first magnet. The output droplet is divided from the first droplet at the fork. As a result, the output droplet comprises the second fluid and at least some of the plurality of magnetic particles.

In other sets of embodiments, a processor or computer-readable medium is configured to perform one or more steps of the above method, or a droplet is a composition of matter produced by one or more steps of the above method.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is a block diagram that illustrates an example picoinjector, according to an embodiment;

FIG. 2A is a block diagram that illustrates an example high-throughput system to exchange fluids in droplets, and thus wash magnetic particles in those droplets, and any targets attached thereto, according to an embodiment;

FIG. 2B is a block diagram that illustrates an example high-throughput microfluidic device to exchange fluids in droplets, and thus wash magnetic particles in those droplets, and any targets attached thereto, according to another embodiment;

FIG. 3 is a flow diagram that illustrates an example method to operate a device in FIG. 2A or FIG. 2B for high-throughput exchange of fluids in droplets containing magnetic particles, according to an embodiment;

FIG. 4 is an image that depicts experimental apparatus in operation for high-throughput exchange of fluids in droplets containing magnetic nanoparticles, according to an embodiment;

FIG. 5 is a graph, with image inset, which illustrates example fluid distribution in droplets of the experimental apparatus of FIG. 4, according to an embodiment;

FIG. 6A is a three panel image that depicts droplet volume changes at a fork in experimental apparatus in operation for high-throughput, continuous exchange of fluids in droplets, according to an embodiment;

FIG. 6B and FIG. 6C are graphs that illustrate example size distribution of droplets of the experimental apparatus of FIG. 6A, according to an embodiment;

FIG. 7 is an image that depicts droplet fluid asymmetry in experimental apparatus in operation for high-throughput exchange of fluids in droplets, according to an embodiment;

FIG. 8A through FIG. 8F are images that illustrates example microparticle migration and separation in experimental apparatus in operation for high-throughput exchange of fluids in droplets containing magnetic microparticles, according to an embodiment;

FIG. 9A is an image that illustrates example dual stage fluid exchange in experimental apparatus in operation for high-throughput exchange of fluids in droplets, according to an embodiment;

FIG. 9B is a diagram that illustrates dual stage fluid exchange implemented in the apparatus of FIG. 9A, according to an embodiment;

FIG. 9C is a graph that illustrates a calibration curve of concentration of dye in known reference droplets, according to an embodiment;

FIG. 9D is a graph that illustrates concentration of dye in output droplets from stage 1 and stage 2 of the equipment of FIG. 9A compared to the concentration of dye in known reference droplets, according to an embodiment;

FIG. 9E is a graph that illustrates comparison of concentration of dye in input droplets to stage 1 compared to concentration of dye in output droplets from stage 2 of the equipment of FIG. 9A, according to an embodiment;

FIG. 10 is a graph that illustrates example migration of magnetic microparticles within a droplet as a droplet streams along the microchannel of FIG. 2A, according to an embodiment;

FIG. 11 is a block diagram that illustrates a computer system upon which an embodiment of the invention may be implemented; and

FIG. 12 illustrates an electronic chip set upon which an embodiment of the invention may be implemented.

### DETAILED DESCRIPTION

A method and apparatus are described for high-throughput washing of magnetic particles and any targets attached thereto in droplets. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of hydrophilic micron-scale (microfluidic) droplets with microscale paramagnetic beads, and oil as a hydrophobic spacer fluid using a particular set of picoinjectors. However, the invention is not limited to this context. In other embodiments, hydrophilic or hydrophobic droplets to various degrees of larger or smaller size with larger or smaller magnetic particles of different materials, including permanently magnetized or superparamagnetic materials, with or without various targets, or different spacer fluids are used with the same or different embodiments of the picoinjectors, such as picoinjectors using salt water electrodes instead of metal electrodes, different apertures, and different approach angles.

### 1. OVERVIEW

Four primary approaches for washing (exchanging fluids in droplets) currently exist. The first approach is called droplet synchronization. This technique involves synchronizing two distinct trains of droplets, one train contains the target to be washed on magnetic beads, the other contains the wash buffer. Droplets are electro-coalesced at a junction while magnetic beads are transferred to the wash droplet via an external magnetic field before splitting. Electro-coalescence involves applying an electric field to break down the surface tension that exists between droplets of the same or different fluids. Droplet synchronization is highly dependent on droplet generation frequency. An emulsion is a mixture of two or more liquids that are normally immiscible with one fluid phase dispersed in the other continuous fluid phase. After such dispersion, it is difficult to synchronize the droplets after they are injected into a microchannel; thus, droplet synchronization is not suitable for re-injecting droplets from emulsions. (Lee et al. 2014). Furthermore, the process is not efficient because droplet synchronization is difficult and error-prone; and, it is difficult to control where the two fluids are initially positioned within each coalesced droplet.

The second approach is called digital droplet washing. This is not a continuous process but requires an array of discrete pads made of metal or dielectric materials which are individually addressable for applying an electric field. Droplets are deposited on the pads and individually subjected to an electric field that weakens the surface tension of the droplet causing the droplets to assume a flatter profile on the pad, in a process called electrowetting. This technique of droplet washing allows for precise droplet movement and washing of magnetic beads using an additional external magnetic field in concert with the electrowetting. Also referred to as electrowetting on dielectric (EWOD), this technique has limitations of scale as the number of washed droplets is limited by the number of pads fabricated on the substrate; and, as stated above is thus not a continuous flow approach to droplet washing. (Sista et al. 2008).

The third approach is called droplet wash and split loop. This technique (described in Strey et al. patent application publication US20110059556A1) pipes droplets (with magnetic beads each bound to a target) and wash droplets into a channel at a one-to-one correspondence. Then the droplets are electro-coalesced at a junction. The droplets are then split on the device or a different device again. An external magnetic field is used to partition beads to one portion of the droplet prior to splitting. Here the wash efficiency is dependent on the volume ratio between the sample droplet and the wash droplet. Synchronization of the two droplets dictates that the droplets are of similar volume and thus a typical wash efficiency is on the order of 50%. Additionally, the droplets are only split after significant amount of time has passed; thus, the two different fluids have likely mixed via diffusion or advection (mechanically driven flow) or convection (thermal driven flow), or some combination. Further washing can be performed by looping through the procedure again until the desired amount of washing has taken place.

The third approach is called magnetic tweezer based bead retention. This technique utilizes a magnetic tweezer apparatus which is an electromagnet with a particular narrow metal core at one or both ends. This technique has been demonstrated in tubing with droplets, but only at low frequencies (˜10 Hz) and requires an electromagnet that is unfavorable for small format lab-on-a-chip devices.

In contrast to the above approaches, the embodiments described herein include a picoinjector (Abate et al., 2010) and a magnet and a fork in a microchannel combined to operate together continuously at high frequencies (for high-throughput) with small footprint. The picoinjector adds a second fluid to a first fluid in a droplet in a laminar flow. The magnet relocates magnetic particles, such as paramagnetic beads, from a portion of the droplet dominated by the first fluid into a portion of the droplet dominated by the injected second fluid before the fluids mix to any great extent by diffusion or advection or any other process. The fork separates the portion with the second fluid and relocated beads from a portion of the droplet with the first fluid and few if any residual beads, again before the fluids mix via diffusion or advection to any great extent.

Picoinjection utilizes electrodes to generate a non-uniform electric field at the junction of a droplet channel and a wash buffer supply channel. As a droplet passes through the junction, the wash buffer, through the aid of applied pressure in addition to electro-coalescence, is drawn into the droplet. At low voltages (0.8-1.0 V controller, ˜40V 40 kHz from inverter) the injected wash buffer remains on the periphery of the droplet where the injection occurs due to long (diffusive) mixing times in the laminar flow regime. This creates a droplet, which is essentially fluid A on one side and fluid B on the other side; half and half if the injected volume equals the original droplet volume.

The picoinjector provides several advantages over droplet coalescence. At least one advantage is that there is no second droplet to synchronize. At least another advantage is that the position of the second fluid immediately after injection is known precisely. At least these advantages are utilized in the various embodiments.

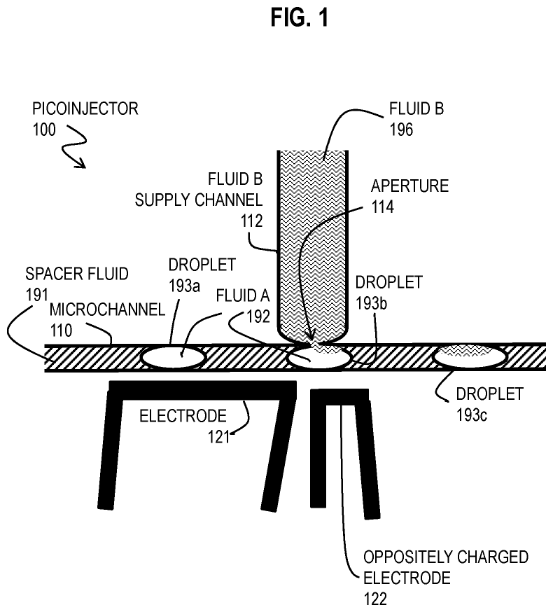

FIG. 1 is a block diagram that illustrates an example picoinjector 100, according to an embodiment. Although a spacer fluid 191, fluid A, droplets 193a, 193b and 193c (collectively referenced hereinafter as droplets 193), and fluid B are included in the drawing to demonstrate operation, these are not part of the picoinjector 100 apparatus itself. The picoinjector 100 incudes a microchannel 110, a fluid B supply channel 112 with an aperture 114 onto a side wall of the microchannel 110, electrode 121 and separate electrode 122. For simplicity it is assumed that the width and depth, and thus the cross sectional area, of the microchannel 110 is constant throughout the portion illustrated in FIG. 1.

During operation, a stream of droplets 193 separated by spacer fluid 191 (indicated by rising diagonal hatching) move through the microchannel 110 under a first pressure (provided by gravity feed, syringe or pump, or some combination). Droplet 193a comprises fluid A 192 (indicated by white space with no hatching). Supply channel 112 is filled with fluid B (indicated by wavy line hatching) at a pressure (provided by gravity feed, syringe or pump, or some combination) that is too small to force fluid B into the microchannel (e.g., less than a sum of the pressure in microchannel 110 and the surface tension of fluid B with the spacer fluid 191 or with droplet 193a of fluid A). Without an electric field produced between electrode 121 and electrode 122, fluid B will not enter the droplets 193a of fluid A. However, when electrode 121 and electrode 122 are oppositely charged with at least a certain voltage capable of reducing the surface tension of droplet 193a, fluid B enters through the aperture 114 into a droplet, as depicted for droplet 193b. After the droplet looses contact with the aperture 114, the droplet has taken on a picoliter volume of fluid B which initially resides on a side of the droplet adjacent to the side wall of the microchannel where the aperture is, as depicted in droplet 193c. The droplet 193c is larger (has a greater volume and therefore occupies a longer section of the channel) by the volume added while the droplet was in contact with the aperture.

By varying the electric field between electrodes 121 and 122, or the pressure on fluid B in the supply channel, or both, the volume injected can be controlled. The electric field can be held constant to inject the same volume into every droplet that passes the aperture, or varied to vary the amount of fluid B injected, or be turned off as a droplet passes to avoid injecting fluid B into that droplet. If held constant, the electrodes can be connected directly to a battery and a resistor to drop the voltage to the desired value without involvement of a controller. In example embodiments described below, the electrodes are charged using an alternating current (AC) electric field (at about 40 kHz) generated using an fluorescent light inverter, as described in Abate et al. 2010.

In various embodiments using the picoinjector, by selecting a distance D from picoinjector to a fork for a given range of flow rates and by applying a small footprint magnet along the microchannel between picoinjector and fork, any magnetic particle and any attached target can be moved into the fluid B portion of the droplet, and the droplet divided, before there is appreciable mixing of the two fluids. This can make the wash much more effective than just 50% each operation. In some embodiments, prior to injection, the magnetic particles are positioned at the portion of the droplet closest to a wall of the microchannel where the picoinjector aperture is located, due to an external magnetic field from either the downstream magnet or a second upstream magnet or both.

### 2. APPARATUS AND SYSTEM FOR HIGH-THROUGHPUT WASHING OF DROPLETS

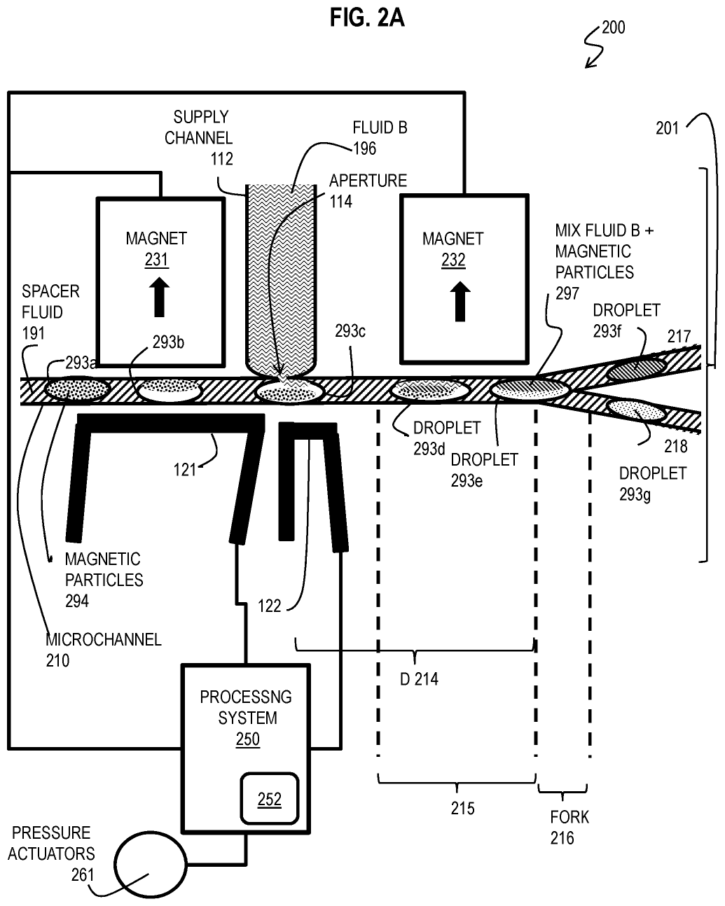

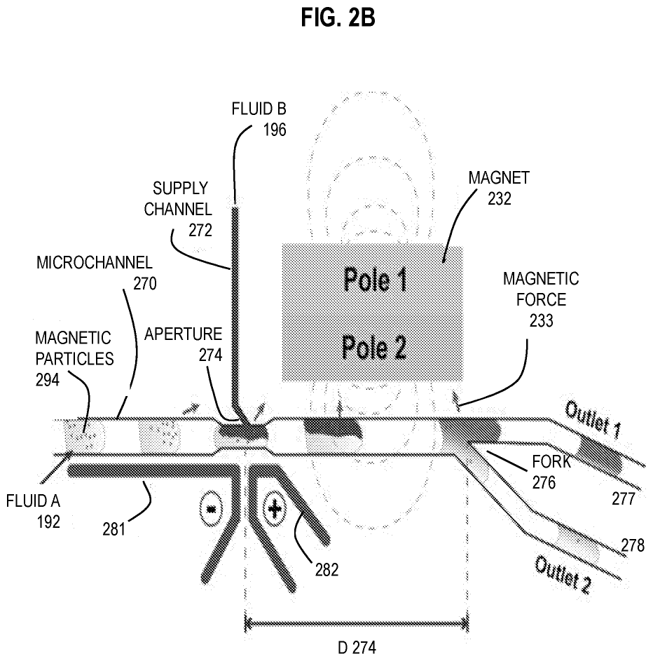

FIG. 2A is a block diagram that illustrates an example high-throughput system 200 to exchange fluids in droplets, and thus wash or remove magnetic particles in those droplets, and any targets attached thereto, according to an embodiment. Although spacer fluid 191, droplets 293a, 293b, 293c, 293d, 293e, 293f, 293g (collectively referenced hereinafter as droplets 293), magnetic particles 294 and fluid B are depicted to illustrate how the system operates, they are not part of the system 200. The system 200 includes processing system 250, with system operation module 252, and pressure actuators 261 along with a microfluidic device 201. The microfluidic device 201 includes the main microchannel 210, fork 216, microchannel branches 217 and 218, magnets 231 and 232, and picoinjector. The picoinjector, such as picoinjector 100, of FIG. 1, includes the supply channel 112 with aperture 114 into a side of the microchannel and electrodes 121 and 122. A distance from the aperture 114 to the fork 216 in the microfluidic device is represented by the symbol D and is used to determine a range of flow rates. A ratio of the cross sectional area of branch 217 divided by the cross sectional area of branch 218 is represented by the symbol R1. At least a section 215 of the distance D is affected by a magnetic field induced by the magnet 232.

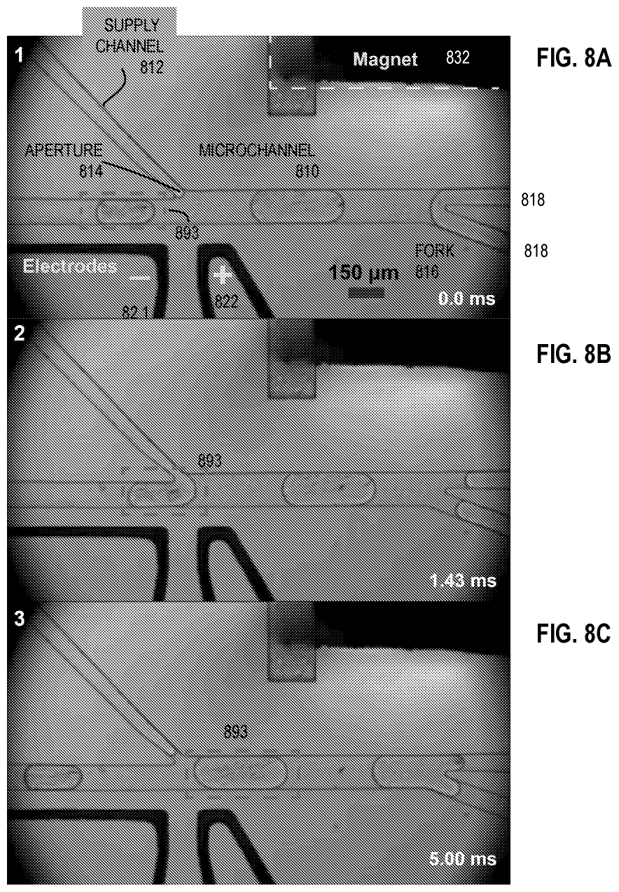

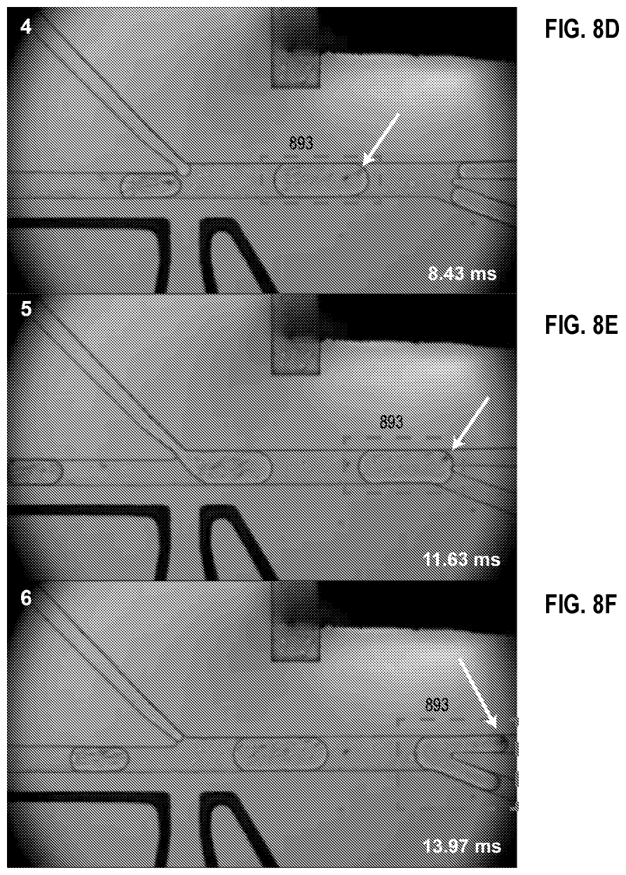

The main microchannel 210 is depicted as having a constant width and is assumed to have a constant dept. However, in some embodiments either the width or the depth or both changes along some sections of the main microchannel 210, as depicted, for example, in FIG. 2B, described below. An advantage of narrowing the cross sectional area of the microchannel 210 in the vicinity of the picoinjector is that the droplet elongates, providing a greater distance to be in contact with the aperture and therefore more opportunity to inject a larger volume of fluid B into the droplet. The result was an observed more reliable/efficient picoinjection. In some embodiments, the cross sectional area of the microchannel is increased immediately downstream of the aperture (compared to the cross sectional area immediately upstream of the aperture) by an amount R3 compared to the upstream cross sectional area, thus the cross sectional area immediately downstream is (1+R3) times the cross sectional area immediately upstream. This can be done to accommodate the extra volume in the flow by virtue of the injected fluid B. The increase is some percentage of the increase in droplet volume injected at the aperture, as depicted in FIG. 8A through FIG. 8F, described below. The volume injected is represented by R2 times the original droplet volume. For example, if the droplets occupy one third of the volume of the stream, e.g., the distance between droplets is about twice the length of the droplet, then the increase in cross sectional area R3=R2/3, i.e., cross sectional area downstream is (1+R2/3) of cross sectional area upstream of aperture. An advantage of this arrangement is that the speed of the stream remains constant even as the volume of each droplet is increased. In other embodiments, other fractions are used. For example, in some embodiments R3=R2, and the cross sectional area (or width if the depth remains constant) of the microchannel 210 immediately downstream of the aperture 114 is (1+R2) times the cross sectional area of the microchannel immediately upstream of the aperture to avoid distortions of the droplet itself.

In some embodiments, the output microchannel branch 217 and the waste microchannel branch 218 have the same cross sectional area as each other and as the main microchannel 210. However, in some embodiments, the cross sectional area (width if the depth remains constant) of the microchannel branches are different from each other or from the input microchannel 210 or both. In some embodiments it is advantageous for the cross sectional area (width if the depth remains constant) of the washed output (first) microchannel branch is about R1 times the cross sectional area (width if the depth remains constant) of the waste output (second) microchannel branch. In some embodiments R1=R2 so that the portion traversing the output microchannel branch 217 can be mostly the injected fluid. Thus, if the injected volume is half the original volume (R2=½), it is advantages for branch 217 to have half the cross sectional area of branch 218 (R1=R2=½). In many of the example embodiments, R2=R1=1. This is advantageous because it ensures the injected volume of fluid B is not overwhelmed by the volume of fluid A from the incoming droplet. In some embodiments, it is advantageous to inject a volume of fluid B that is greater than the original volume of the droplet, so R2 is greater than 1, and R1=R2>1.

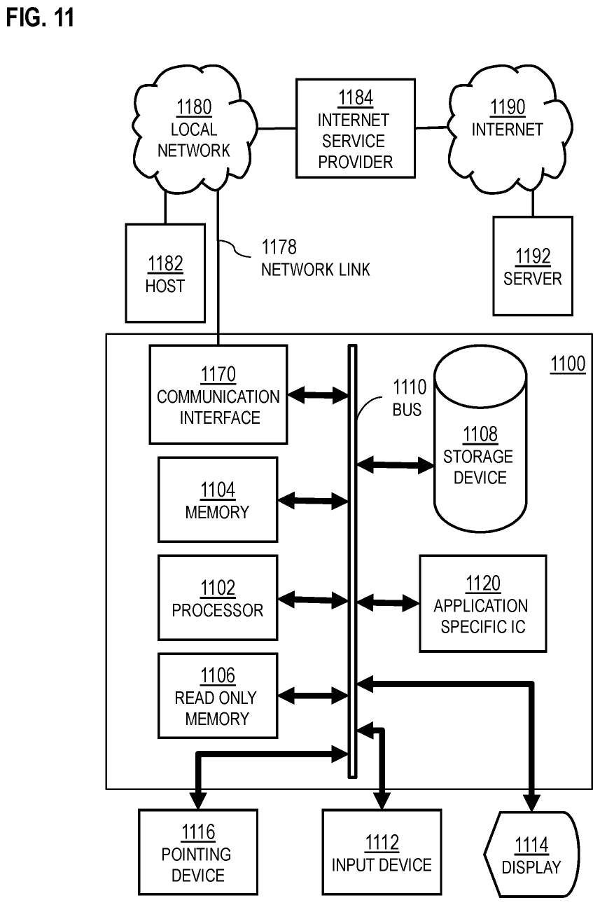

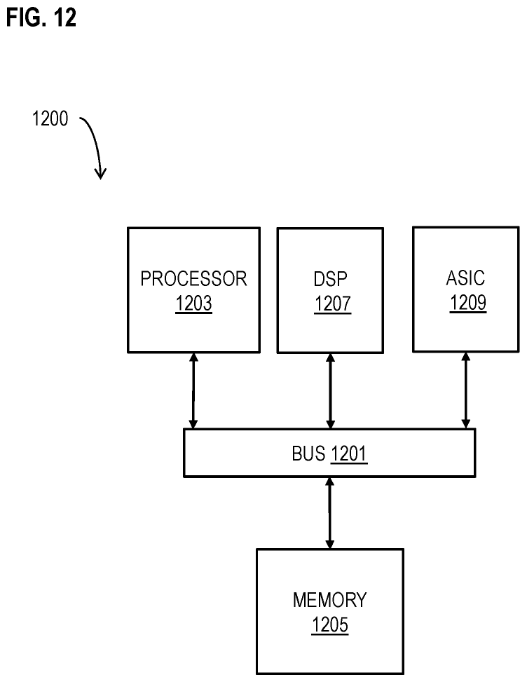

The processing system 250 is a computer system or electronic chip set as descried below with reference to FIG. 11 and FIG. 12, respectively. The operation module 252 controls the timing and voltage difference set up between electrodes 121 and 122, controls the pressure actuators 261, if any, and controls the downstream magnet 232 and optional upstream magnet 231, if those are electrically controlled. In some embodiments, one or both magnets 131 and 132 are permanent magnets; and, the control of permanent magnets by operation module 252 is omitted. In some embodiments, the permanent magnets are moveably mounted to the substrate, and the operation module 252 operates a motor to move the magnet 131 or 132 or both closer or farther from the microchannel 210. The pressure actuators 261 (such as pumps, motors for motorized syringes, or valves for a gravity or capillary feed column, or other mechanisms known in the art, or some combination) are in fluid communication (not shown) with the microchannel 210 and supply channel 112 and any collection reservoirs (not shown) downstream of branches 217 and 218. The pressure actuators cause a pressure to be exerted on the stream of spacer fluid 191 and droplets 293 and the same or different pressure on the fluid B in supply channel 112 and any pressure exerted on any collection reservoirs (not shown).

During operation, initial droplets include fluid A and a plurality of magnetic particles (indicted by dark stippled fill pattern) with any targets attached thereto, as depicted for droplet 293a. The magnetic particles move under the influence of any external magnetic field produced by the downstream (first) magnet 232 and, optionally, the upstream (second) magnet 231.

If the magnetic particles are already magnetized they have a tendency to aggregate rather than to stay dispersed in the fluid A of the droplet. Such particles commonly consist of two components, a magnetic material, often iron, nickel and cobalt, and a chemical component that has functionality. While nanoparticles are smaller than 0.5 micron in diameter (typically 5-500 nm), the larger microbeads are 0.5-500 microns in diameter. Magnetic nanoparticle clusters that are composed of a number of individual magnetic nanoparticles are known as magnetic nanobeads with a diameter of 50-200 nm. Magnetic nanoparticle clusters are a basis for their further magnetic assembly into magnetic nanochains. An advantage of particles of paramagnetic materials is that they do not adopt a magnetized state absent an externally applied magnetic field; and, thus stay dispersed within the fluid of their droplet (e.g., dispersed within fluid A of droplet 293a). When magnetized in a external magnetic field, the particles then move toward the stronger field (e.g., toward the nearest pole of the external magnet that induced the magnetic state) before the particles aggregate. Paramagnetism is a form of magnetism whereby certain materials are weakly attracted by an externally applied magnetic field, and form internal, induced magnetic fields in the direction of the applied magnetic field. In contrast with this behavior, diamagnetic materials are repelled by magnetic fields and form induced magnetic fields in the direction opposite to that of the applied magnetic field. Some materials show induced magnetic behavior that follows a Curie type law but with exceptionally large values for the Curie constants. These materials are known as superparamagnetic. An advantage of superparamagnetic particles is that they respond more strongly to the externally applied magnetic field and move faster to the inducing magnet. They are characterized by a strong ferromagnetic or ferrimagnetic type of coupling into domains of a limited size that behave independently from one another. The bulk properties of such a system resemble that of a paramagnet, but on a microscopic level they are ordered. The materials do show an ordering temperature above which the behavior reverts to ordinary paramagnetism. When magnetized in a external magnetic field, diamagnetic particles then move away from the stronger field (e.g., away from the nearest pole of the external magnet that induced the magnetic state) before the particles aggregate.

Any magnet may be used for the downstream magnet 232 or optional upstream magnet 231. The direction that the magnet moves the magnetic particles used in the droplets 293 is given by the arrow. Thus the magnet is on the same side of the microchannel 210 as the aperture 114, as depicted in FIG. 2A and following figures, when the magnetic particles are magnetized or paramagnetic. However, if the magnetic particles are diamagnetic, then the magnets 131 or 132 or both are on the opposite side of the microchannel 210 from the aperture 114. In either the magnetized particle embodiment or the paramagnetic particle embodiment or the diamagnetic particle embodiment, the downstream magnet 132 is configured to introduce a magnetic field into the microchannel between the picoinjector and the fork to move magnetic particles in a droplet toward the same side of the microchannel as the aperture, also called the “aperture side” or the “first side” herein.

Any magnet used in the art may be used as either or both magnets 131 and 132, including magnetic tweezers and other electromagnets. An advantage of certain permanent magnets is that they can be fabricated small enough (on the order of millimeters in each dimension) to be fully in place in a microchip and do not require a power source or control by the operation module 252. An example permanent magnet with this capability, as used in the experimental embodiments below, is a rare earth Neodymium magnet. In some embodiments, either magnet 131 or 132, or both, are composed of a plurality of magnets placed side by side, or stacked, on the same side of the microchannel.

In some embodiments, upstream magnet 231 is also configured to move the magnetic particles toward the first side of the microchannel (i.e., the side with the aperture 114) but does so upstream of the aperture 114 to pre-position the magnetic particles in the droplet. This has the advantage, in some embodiments, of reducing the time it takes (and thus the distance needed of the available distance D) to move the magnetic particles back to the aperture side of the microchannel 210 after fluid B is injected at the picoinjector. In some embodiments, the microfluidic device excludes upstream magnet 231.

The stream of droplets and spacer fluid 191 is caused to move at a flow rate such that a droplet covers the distance D in a migration time T_{M} sufficiently long for magnetic particles to migrate across the droplet, yet short compared to the time it takes fluid B to mix with fluid A within a droplet. In general, T_{M} is short compared to a time for a first fluid (A) to mix with the second fluid (B) in the droplet; and, T_{M} is long compared to a time for the magnetic particles to move in the magnetic field of the downstream magnet 232 at least a tenth of the width of the microchannel 210 in a section 215 between the electrodes 122 and the fork 216 where magnet 232 exerts a magnetic field. This time can be easily determined by a person of ordinary skill in the art by experimentation. Example experiments to do so, among others, are described below in the section on example embodiments.

The droplets 293 depicted in FIG. 2A can be considered different droplets in a snapshot of the system during operation. Alternatively, the droplets can be considered to be the same droplet at different times as the droplet traverses the device left to right through the microchannel 210 and is split at fork 216 to have one portion exit on output microchannel branch 217 (also sometimes called the “first branch” or “output 1 channel” herein) and the other portion exit on waste microchannel branch 218 (also sometimes called the “second branch” or “output 2 channel” herein).

Initially, a droplet 293a enters the microchannel 210 with magnetic particles 294 dispersed in a fluid A. In the magnetic field of upstream magnet 231, if present, the magnetic particles move in the direction of the arrow to the side of the microchannel where the aperture of the picoinjector is, as depicted in droplet 293b. At the aperture 114 of the picoinjector, when the electrodes are oppositely charged by the operation module 252 and sufficient pressure is applied to the supply channel by actuators 261, fluid B, represented by the wavy hatching, pours as a laminar flow into the droplet, pushing fluid A and the magnetic particles away from the side of the droplet on the aperture side, as depicted in droplet 293c, and increasing the volume of the droplet, but not yet mixing with fluid A. In the presence of the field of the downstream magnet 132, the particles move from the portion of the droplet occupied by fluid A into the portion of the droplet occupied by fluid B. The portion of the droplet with both fluid B and the magnetic particles is indicated by the close downward diagonal hatching in droplet 293d. By the time the droplet arrives at the fork 216, as depicted in droplet 293e, it is desirable that the portion of the droplet on the aperture side of the microchannel comprises both fluid B and many of the magnetic particles (close downward diagonal hatching), leaving few, if any, magnetic particles in the portion of the droplet having fluid A (small dotted fill).

At the fork 216 the aperture side portion of the droplet 293e is severed from the opposite side portion. The aperture side portion traverses the output (first) microchannel branch 217 as reduced size droplet 293f with predominately fluid B and a majority of the magnetic particles. The opposite side portion traverses the waste (second) microchannel branch 218 as reduced size droplet 293g with predominately fluid A and relatively few magnetic particles. Thus the magnetic particles, and any targets attached to them, are effectively “washed” with fluid B, which has been effectively exchanged for fluid A. The washed droplets in branch 217 are collected for further use, e.g., in another stage of a microfluidic device or system, or in a basin for harvesting. Similarly, the waste droplets in branch 218 are disposed of, either by being discarded or processed in another stage of a microfluidic device or system. In some embodiments, the waste droplets are useful as droplets from which the magnetic particles have been largely removed.

In some embodiments, the droplets are of constant size and composition, the pressure on the microchannel is constant, the electrodes are consistently charged and pressure in supply channel is constant so that every droplet that passes the aperture 274 has the same volume of fluid B injected. In such embodiments, the operation module 252 in processor 250 can be omitted.

FIG. 2B is a block diagram that illustrates an example high-throughput microfluidic device 202 to exchange fluids in droplets, and thus wash magnetic particles in those droplets, and any targets attached thereto, according to another embodiment. Although droplets, magnetic particles 294, fluid A (light gray) and fluid B (dark grey) are depicted to illustrate how the device operates, they are not part of the microfluidic device 202. The microfluidic device 202 includes the main microchannel 270, fork 276, microchannel branches 277 and 278, magnet 232, and picoinjector. The picoinjector includes the supply channel 272 with aperture 274 into a side of the microchannel 270 and electrodes 281 and 282. The architecture of the picoinjector channel may depend on various factors such as: Magnetic particle size, magnetic particle type, position of the magnet relative to the picoinjection supply channel, the diameter of the aperture of the picoinjection channel. It has been observed, especially for small (<−5 μm diameter) magnetic particles and superparamagnetic particles that immediately after injection and under the influence of a magnetic field that particles can traverse into the picoinjection channel. This is undesirable as this could lead to cross-contamination between droplets or partitions. In order to remedy this, the architecture of the picoinjection channel might be modified. Examples of modifications to the channel include but are not limited to changing the aperture size of the picoinjection channel (diameter/height) or the angle of incidence of the picoinjection with the main (droplet containing) channel preferably such that the picoinjection channel is oriented away from the magnet. Thus, the narrow slanted supply channel 272 depicted was found to be advantageous for smaller superparamagnetic microparticles and nanoparticles because such small particles can be pulled into larger or perpendicular supply channels by the magnetic field of a downstream or upstream placed magnet. The use of a narrow and slanted supply channel 272 avoids tedious or fruitless efforts to optimally position the upstream or downstream magnet.

A distance 274 from the aperture 274 to the fork 276 in the microfluidic device is represented by the symbol D and is used to determine a range of flow rates. The magnetic field is represented by dashed curved gray lines emanating from the magnet 232, and the force 233 imposed on the magnetic particles 294 by the downstream magnet 232 are represented by arrows and extend upstream of the aperture 274. In this embodiment, the differences from the device 201 in FIG. 2A are that there is only the downstream magnet, the width of the microchannel 270 is decreased in the vicinity of the aperture 274 of the picoinjector, the angle between branches 277 and 278 is different, the electrodes are differently shaped and particularly charged, with electrode 281 a cathode (negatively charged) and electrode 282 an anode (positively charged), and the supply channel 272 is the same width as the aperture 274 and approaches the microchannel 270 non-perpendicularly. The operation, however, is similar to that described above for FIG. 2A.

Although processes, equipment, and components are depicted in FIG. 2A and FIG. 2B as integral blocks in a particular arrangement for purposes of illustration, in other embodiments one or more processes or components, or portions thereof, are arranged in a different manner, on the same or different computers or microfluidic chips, or are omitted, or one or more different processes or components are included on the same or different microfluidic chips.

For example, in some embodiments the components described above for the microfluidic device 201 or 202 constitute one stage of a system having multiple stages. The additional stages can be on the same substrate or in separate devices that are chained together, taking the droplets from either or both branches 217 and 218 (or 277 and 278) as input to the main microchannel of the next stage. In some embodiments, the output from one branch, e.g., from branch 217 or 277 is fed back into the same stage for a second round of washing. Thus, the described device can be implemented in serial so as to obtain higher fluid exchange efficiency. This involves the duplication of integral components such as the injection channel, magnet, and splitting junction in each of one or more additional stages. It is advantageous if the fluid resistance is matched between the first branch (e.g., 217 or 277) and the remaining second or more stages of the described device. This is achieved by measurement of channel lengths which are used as a proxy for fluidic resistance. Modelling the fluid path as an equivalent circuit allows for the application of Kirchoffs law to match the fluidic resistances, normalizing the flow rates, as described by Oh et al., 2012. Such models are readily available for public use, such as COMSOL available as in folder microfluidics-module of World Wide Web domain comsol in domain extension corn.

### 3. METHOD FOR HIGH-THROUGHPUT WASHING OF DROPLETS

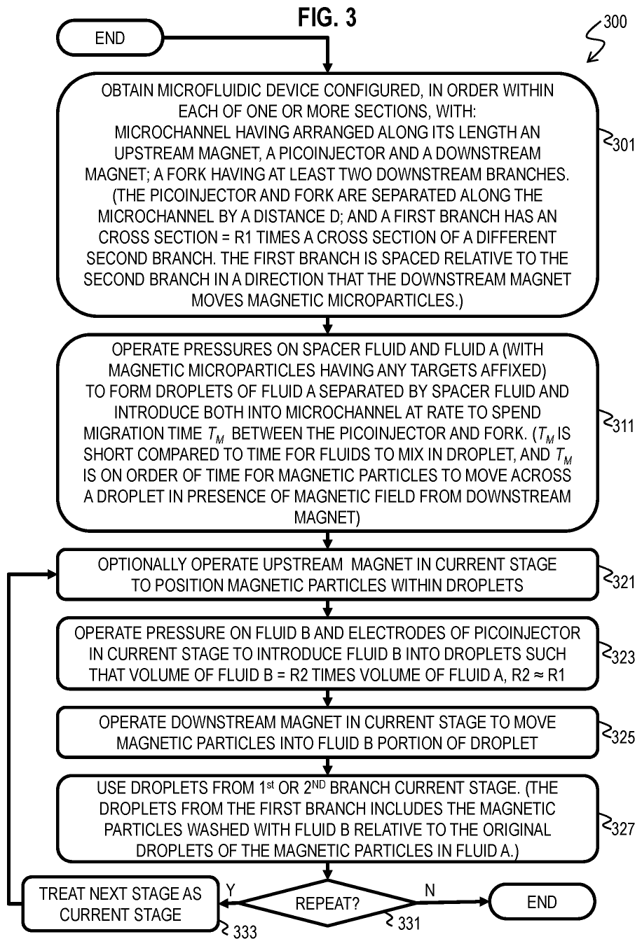

FIG. 3 is a flow diagram that illustrates an example method 300 to operate a device depicted in FIG. 2A or FIG. 2B for high-throughput, continuous exchange of fluids in droplets containing magnetic particles, according to an embodiment. Although steps are depicted in FIG. 3, as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 301 a system is obtained, such as by purchase or fabrication or reconfiguration or reuse, with a microfluidic device that is configured as depicted in FIG. 2A as device 201 or in FIG. 2B as device 202, or equivalents thereof. The microfluidic device of such a system is recognizable as follows. The device includes a microchannel (e.g., 210 or 270), a plurality of microchannel branches comprising a first microchannel branch (e.g., 217 or 277) and a different second microchannel branch (e.g., 218 or 278), a fork (e.g., 216 or 276) comprising a junction between the microchannel upstream of the fork and the plurality of microchannel branches downstream of the fork, a picoinjector disposed along the microchannel, the picoinjector comprising a supply channel (e.g., 112 or 272) connected to the microchannel by an aperture (e.g., 114 or 274) on a first side of the microchannel and a pair of electrodes (e.g., 121 or 281 and 122 or 282) on an opposite side of the microchannel, wherein the aperture is a distance D (e.g., 214 or 274) upstream of the fork. The device also includes a first magnet (e.g., 232) disposed adjacent to the microchannel between the picoinjector and the fork. The first branch is distinguished from the second branch because the first branch is spaced relative to the second branch in a direction that the downstream magnet moves magnetic particles. Stated another way, the first branch is on the same side as the aperture of the picoinjector or shares a channel wall with the aperture of the picoinjector. In some embodiments, the ratio of the cross sectional areas of the branches affect the operation. For some such embodiments, it is useful to note the ratio R1 of the cross sectional area of the first branch divided by the cross sectional area of the second branch.

In step 311, pressure is applied on spacer fluid and fluid A (with magnetic microparticles having any targets affixed) to form droplets of fluid A separated by spacer fluid and introduce both into microchannel at a rate to spend migration time T_{M} between the picoinjector and fork. Thus step 311 involves causing a stream of a plurality of droplets separated by a spacer fluid to flow through the device, wherein each droplet of the plurality of droplets comprises a first fluid with a plurality of magnetic particles. T_{M} is short compared to time for fluids to mix in droplet, and T_{M} is on the order of a time for magnetic particles to move across a droplet in presence of the magnetic field from downstream magnet. For example, T_{M} is short compared to a time for the first fluid to mix with the second fluid in the droplet and long compared to a time for the magnetic particles to move in the magnetic field of the first magnet at least a tenth of the width of the microchannel between the electrodes and the fork. In experimental embodiments described below, T_{M} is on the order of 10 milliseconds (ms, 1 ms=10⁻³ seconds). For D on the order of 100 microns, an example speed is about 1 centimeter (cm, 1 cm=10⁻² meters) per second (cm/s). So, a pressure sufficient to move a stream of spacer fluid and droplets on the order of 1 cm/s is applied during step 303. For various embodiments, the range of migration times T_{M} is from about 1 to about 100 milliseconds and the range of distances D is from about 100 to 1000 microns, so the range of speeds is about 0.1 cm/s to about 100 cm/s; and, pressures sufficient to move the stream at such rates are used. It is noted here that for droplets spaced in the microchannel on the order of one droplet every 100 to 1000 microns, the above range of speeds corresponds to rates of droplet processing in a range from about 1 to about 10,000 droplets per second, and thus can achieve sample processing rates up to about 10 kiloHertz (kHz, 1 kHz=10³ samples per second) for closely spaced droplets (100 microns) moving at high speeds (100 cm/s).

In some embodiments, the components recited above for the microfluidic device are repeated as additional stages downstream on the same substrate or in a second microfluidic device or the output from either branch is fed back as input to the microchannel (e.g., 210 or 270). The following steps 321 through 327 are repeated for each stage of washing.

In step 321, an upstream magnet (e.g., 131) is operated to position magnetic particles within the droplet. In some embodiments, the upstream magnet is a permanent magnet and step 321 is performed inherently without input from an operation module 252. In some embodiments, experimentation determines where the magnetic particles are best positioned by upstream magnet prior to injecting fluid B (the second fluid). In the example embodiments, it was found advantageous to positon the magnetic particles on the same side as the aperture of the picoinjector to get a fast response from the particles after injecting the fluid B. Thus, in such embodiments, step 321 involves introducing from the second magnet a magnetic field into the microchannel upstream of the picoinjector to move magnetic particles in the first droplet toward the aperture side of the microchannel before injecting the volume of the second fluid into the first droplet. In some embodiments, there is no upstream magnet; and, step 321 is omitted; and, the method moves directly to step 323.

In step 323, the picoinjector is operated to introduce fluid B (the second fluid) into at least one droplet (the first droplet), such that the volume of fluid B=R2 times the volume of fluid A (the first fluid) in the droplet. Thus step 323 involves supplying the second fluid (fluid B) to the supply channel under pressure and applying a voltage difference to the pair of electrodes when a first droplet of the plurality of droplets is in contact with the second fluid at the aperture to inject through the aperture a volume of the second fluid into the first droplet. In some embodiments, the amount of fluid B injected, given by R2, is related to the relative size R1 of the branches downstream of the fork. For example, R2=R1. In some embodiments, the amount of fluid B injected, given by R2, is related to (based on) the relative size of the main microchannel (e.g., 210) immediately upstream and downstream of the aperture of the picoinjector, given by R3. For example, R3=R2/f, where f is the fraction of the stream occupied by droplets, thus R2=f*R3. When operated under different conditions, the value of f may change even as the value of R3 is set for the device; yet, the pressure or voltage difference can be adjusted so that R2=f*R3. In some embodiments in which the desired operating conditions are known, the device can be obtained in step 301 that has R1 and R3 set so that both conditions are satisfied simultaneously, i.e., there is a fraction f such that R2=R1=f*R3.

In step 325, the downstream magnet is operated to move magnetic particles in the droplet into a fluid B portion of the droplet nearest to the side wall of the microchannel that has the aperture. In some embodiments, the downstream magnet is a permanent magnet and step 325 is performed inherently without input from an operation module 252. Thus step 325 involves introducing from the downstream magnet a magnetic field into the microchannel between the picoinjector and the fork to move magnetic particles in the first droplet toward the aperture side of the microchannel.

In step 327, the droplets at least from the first branch on the aperture side of the device (e.g., branch 217 or 277) are used. These droplets include the magnetic particles washed with fluid B relative to the original droplets in which the magnetic particles are dispersed in fluid A. For example, these droplets are collected in a reservoir or fed to another microfluidic device or process. In some embodiments, it is desirable to use a droplet of fluid A from which the magnetic particles have been removed. In such embodiments, the droplets are used from a different second branch (e.g., branch 218 or 278) that is not on the aperture side of the device.

In step 331, it is determined whether magnetic particle washing (or magnetic particle removal) is to be repeated in another stage. If so, the droplets from one branch or the other or both are directed to the next stage, which becomes the current stage of washing or removal and steps 321 to 327 are repeated. If not, the process ends.

### 4. EXAMPLE EMBODIMENTS

Several experimental embodiments were constructed and filmed to demonstrate the effects described above. A droplet microchannel 210, upstream of which is a flow-focusing junction of aqueous fluid A (and any magnetic beads as magnetic particles) and oil spacer fluid, meets a supply channel 112 with wash buffer (fluid B) at an aperture 114. Immediately next to this aperture and perpendicular to the supply channel are two electrodes 121 and 122. Some distance D away, from the aperture is a channel bifurcation (fork 216) leading to two outlets branches: outlet 1 (217) carrying droplets with mostly fluid B with most of the magnetic beads, if any in the input droplet; and, outlet 2 (218) carrying droplets with mostly fluid A (input) and very few magnetic beads, if any in the input droplet. In these experiments, the magnetic particles are magnetic beads without targets affixed thereto; and in some experiments, designed to demonstrate persistence of fluid separation after picoinjection to determine T_{M}, no magnetic particles are used at all.

In these experimental embodiments, during injection, the magnetic beads present in the droplet will disperse slightly from the injection point due to forces of injection. Initially the beads will be pushed away from the newly created laminar interface. After a short time however, the beads will be pulled according to the magnetic force across the interface into the wash buffer portion of the droplet. After the beads have aligned to the wash buffer portion and before the droplet has sufficient time to mix according to diffusion, or other processes, the droplet is physically split at the fork downstream. The resultant droplets are a ‘waste droplet’ containing mostly fluid A and any magnetic beads that did not transit the laminar interface in time, and a ‘washed droplet’ containing the vast majority of magnetic beads in a high concentration of fluid B. Effectively the magnetic beads have been transferred from a droplet with a high concentration of fluid A (no fluid B) to droplet with a high concentration of fluid B and a low concentration of fluid A.

The concentration of fluid A in the washed droplet will be fluid A that had time to diffuse and or, through internal droplet advective or convection fluid movements, migrate to above the horizontal axis formed by the splitting point at the fork, or buffer that was above the splitting point immediately after injection. Both of these effects can be minimized in the following ways. 1) To minimize the amount of wash buffer that remains above the horizontal axis formed by the split point at the fork, the amount of injected buffer should be increased to cause the laminar interface after injection to be at or below the width of the split point in the fork. 2) To minimize the amount of diffusive mixing occurring across the laminar interface the droplet flow rate can be increased to minimize the amount of time (T_{M}) to traverse the distance D prior to physical splitting at the fork. This however will decrease the amount of time allowed for the magnetic beads to traverse the laminar interface and extend above the split point of the fork, and therefore should be selected carefully for the particular fluids, drop sizes and magnetic particles used, as demonstrated in the various experimental embodiments below.

### 4.1 Microfluidic Device Fabrication.

The experimental microfluidic devices were fabricated in transparent polydimethylsiloxane (PDMS) according to soft lithography techniques. Inlets and outlets and magnet positions are cut from the PDMS slab and the device is then bonded to glass slide to close and seal the channels. Fluidic channels are treated with AQUAPEL™ (from PPP Industries of Tuakau, New Zealand) to make them hydrophobic. Electrode channels are treated with (3-Mercaptopropyl)trimethoxysilane (MPTMS) to make them wettable to low temperature solder. The device is placed on a hotplate at 215° C. for at least ten minutes, prior to inserting small segments of low temperature indium-tin (52% indium, 48% tin) solder into the electrode inlets Immediately after, small copper wire is placed into the still molten electrode ports to provide attachments to electronic power supply. Electrode copper wire connections can be stabilized using an epoxy overlay to minimize breakage of the wire. Other implementations of the electrode configuration exist, including pre-fabricating electrodes on the substrate and aligning the microfluidic channels to these existing electrodes. Alternatively, salt water electrodes can be used by filling channels with high salt solution and energizing with a power supply connected to the syringe needle in direct contact with the salt water solution. At least one magnet is placed extremely close to the droplet microchannel on a downstream side of the supply channel, which magnet creates a high gradient magnetic field in the region of the microchannel between the aperture and the fork. Specifically, in the experimental embodiments, at least one rare earth Neodymium magnet was placed immediately adjacent to the microchannel along a length of the microchannel in a range from 100-500 microns.

4.2 Washing Magnetic Beads with Food Coloring.

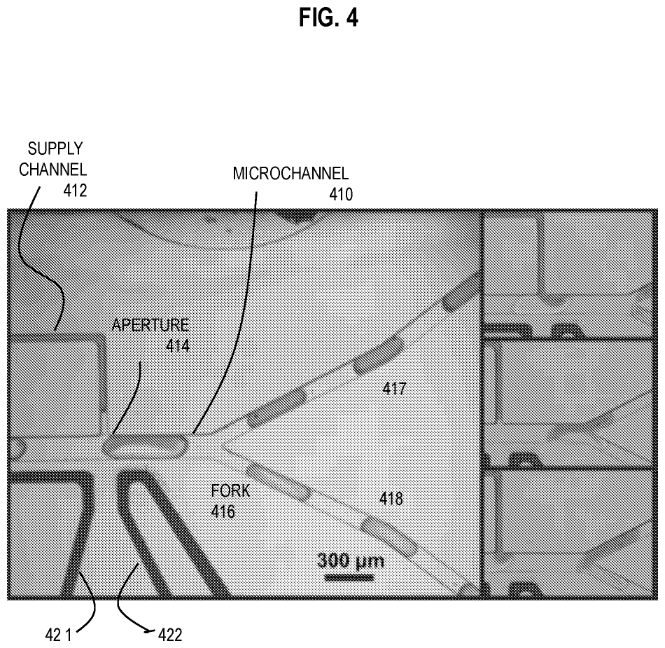

FIG. 4 is an image that depicts experimental apparatus in operation for high-throughput exchange of fluids in droplets containing magnetic nanoparticles, according to an embodiment. Red food coloring (dark gray) was used as the wash buffer (fluid B) to aid in the interface visualization and extent of mixing that occurs between injection and splitting. Incoming clear droplets with 22 micron paramagnetic beads are subjected to picoinjection of red food coloring and split into two steams of droplets, washed droplets in first branch above and waste droplets in second branch below. Washed droplets are very dark indicating a composition of mostly injected buffer; whereas, waste droplets are light due to some wash buffer with the original clear fluid. The three insets to the right depict laminar flow of wash buffer into a droplet (top), the start of the splitting of the same droplet with laminar separation of dark buffer and original clear fluid at the fork (middle), and the end of the splitting of that same droplet (bottom).

### 4.2 Washing Quantification Using Fluorescence

The extent of washing has also been quantified by fluorescence. Incoming sample droplets were loaded with ˜100 milliMolar (mM, 1 mM=10⁻³ Molar) Fluorescein tracer as fluid A, but no magnetic particles. The wash buffer (fluid B) had no Fluorescein. After wash buffer picoinjection, washed droplets, and waste droplets, and split original droplets (obtained by removing the voltage difference between the electrodes) were collected in a single reservoir and imaged using a fluorescent microscope on a hemacytometer. Droplet images were processed by converting to grayscale followed by passing the images through a circle detection algorithm. The algorithm looks for circles by finding bright-dark edges/boundaries in the image. After circle detection, the intensity of the circle is measured by averaging over the pixels in a central portion of each detected circle individually. From this, the fluorescence intensity is graphed as a distribution for the population of droplets. The difference in fluorescence intensity indicates minor transfer of fluorescent species from the sample droplet to the newly created wash droplet.

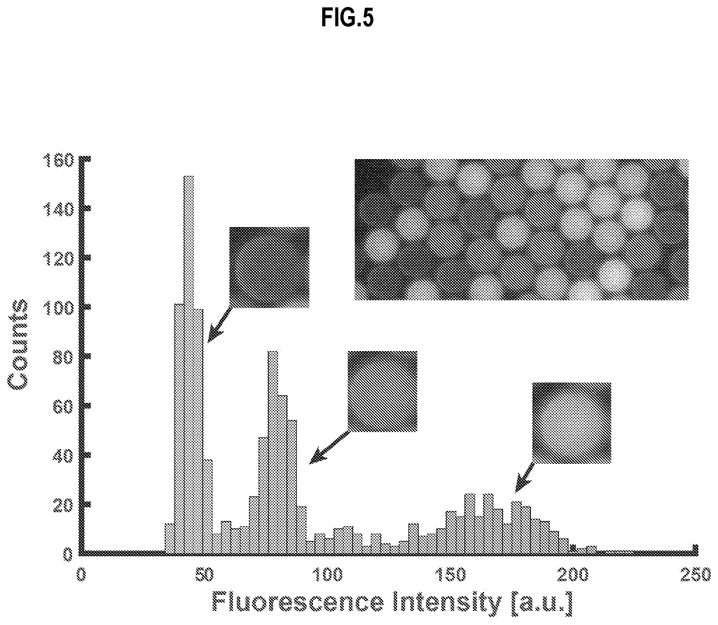

FIG. 5 is a graph, with image inset, which illustrates example fluid distribution in droplets of the experimental apparatus of FIG. 4, according to an embodiment. The inset shows the collection of droplets from all three populations that were subjected to the circle detection algorithm. The lighter circles are droplets with greater fluorescence intensity. The graph is a histogram. The horizontal axis indicates fluorescence intensity in arbitrary units (greatest for the split original droplets, and least for the washed droplets), binned. The vertical axis indicates the number of droplets identified for each binned intensity value. Three population peaks are visible: 1) Un-injected (electrode OFF) droplets [far right], 2) Injected waste (outlet 2) [middle], and 3) injected washed (outlet 1) [left]. Such data can be used to tune the device to further the difference between the washed and waste droplets, by varying the injection pressure, voltage difference, and speed of the stream for a given aperture to fork distance D.

### 4.3 Droplet Volume Control During Washing

This experiment was conducted to demonstrate that the volume injected into the droplet is highly controllable, and that the splitting process can be designed to retain the original volume of the input droplets due to a careful balance of flow rates and pressures. Bromophenol blue (BPB) dye was used as a light sensitive dye for droplet tracking in both the original droplets of fluid A and the wash buffer fluid B. Bromophenol blue exhibits maximum absorptivity at approximately 590 nanometers. Imaging was performed using a 590 nanometer bandpass filter 10 nanometer FWHM (FB590-10, Thorlabs of Newton, N.J.). No magnetic particles were used in this experiment. A high speed video of injection and splitting was taken showing the increase and subsequent decrease of volume for the droplets. The droplets return to the same approximate volume after splitting. All images were analyzed using image analysis correlated to a known feature size. The droplet area was integrated through the channel height to obtain the approximate droplet volume.

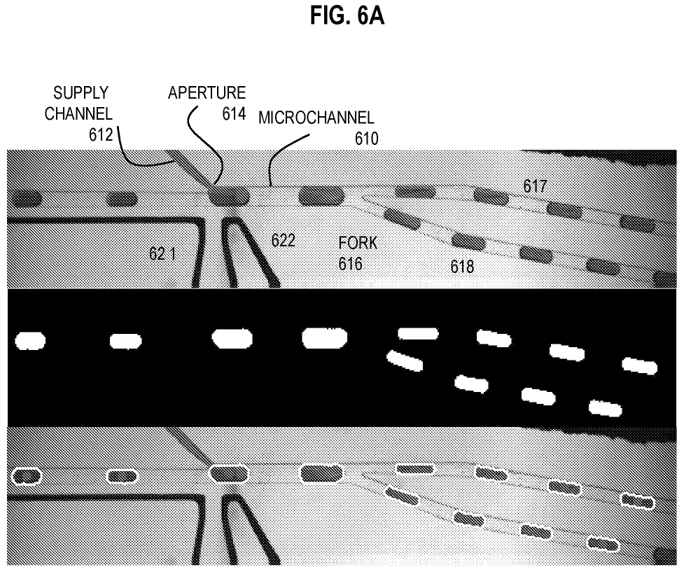

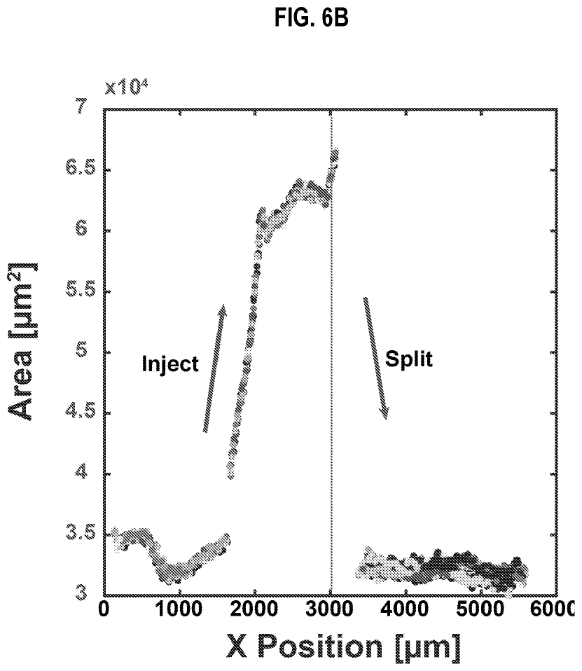

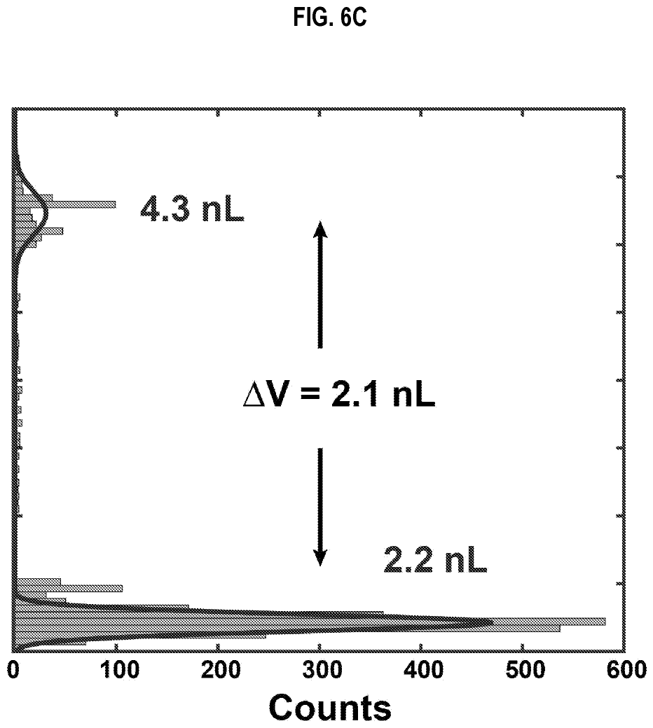

FIG. 6A is a three panel image that depicts droplet volume changes at a fork in experimental apparatus in operation for high-throughput exchange of fluids in droplets, according to an embodiment. The top panel is the original image showing the original droplets on the left in the microchannel 610; the injected droplets between the aperture 614 of the supply channel 612 for the picoinjector (which includes electrodes 621 and 622), and the fork 616; and, the droplets in the two branches 617 and 618. In this case R2=R1=1 and R3>0. Thus the droplet doubles in size (width by (1+R3) since the channel increases in width after the aperture, and length, but depth is constant). The middle panel is a binary mask made by masking out to zero (OFF) all pixel that do not contain the blue of the dye and assigning the maximum value (ON) to all pixels that do show the proper blue color. This mask is used to determine volume by multiplying the number of ON pixels in each droplet by the depth of the channel. The pixel size is determined based on a known standard distance in the image (not shown). The bottom panel shows that the outline of the ON pixels maps well to the outline of each droplet.

After the volume of each droplet is obtained, it is plotted against distance along the device and as a histogram. FIG. 6B and FIG. 6C are graphs that illustrate example size distribution of droplets of the experimental apparatus of FIG. 6A, according to an embodiment. FIG. 6B plots area of the droplet (from the middle panel of FIG. 6A) in microns squared along the vertical axis and the distance along the image in microns along the horizontal axis. The original droplets travel in along the microchannel 610 from 0 to about 1700 microns where the aperture 614 is located and droplet volume is rather constant at about 3 to 3.5 microns squared. Downstream of the aperture for about the width of the droplets, the volume increases as fluid B is injected. From the point at which the droplet breaks with the aperture to the split, about 2000 to 3000 microns, the size is relatively constant with an area of about 6 to 6.5 microns squared. After the split at about 3500 microns, both the washed and waste droplets have the original size at 3 to 3.5 microns squared. A histogram of the number of droplets observed with each volume is shown in FIG. 6C. The horizontal axis indicates count in number of droplets, and the vertical axis indicates area in microns squared. The volume in nanoliters (nL, 1 nL=10⁻⁹ liters) is obtained by multiplying the area of a peak in FIG. 6C by the depth of the channel and converting cubic microns to nanoliters. There is a strong population peak at the original and final droplet volumes of about 2.2 nL, and a second peak at 4.3 nL for the engorged droplets between the aperture 614 and the fork 616. The approximate change in volume is ˜2.1 nanoliters; but, the output washed droplet volume is about the same as the input droplet volume.

### 4.4 Fluid Asymmetry in Injected Droplets

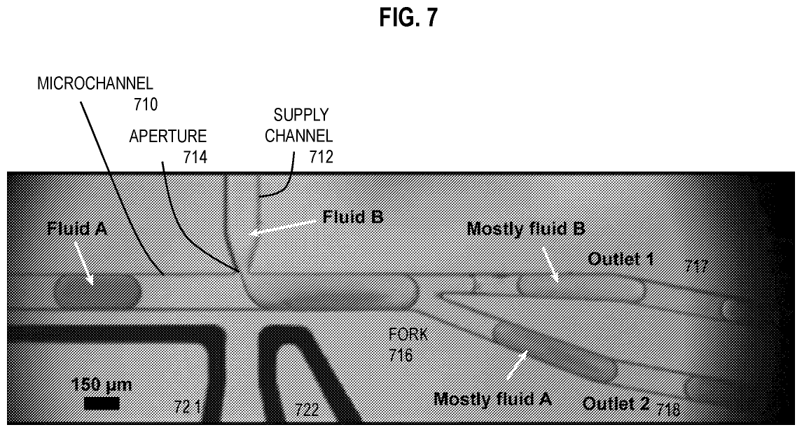

Injection asymmetry was verified using a high speed camera and bromophenol blue (BPB) dye. FIG. 7 is an image that depicts droplet fluid asymmetry in experimental apparatus in operation for high-throughput exchange of fluids in droplets, according to an embodiment. A picoinjector includes supply channel 712 and aperture 712 and electrodes 721 and 722. The microchannel 710, fork 716 and output 1 and output 2 branches 717 and 718, respectively, are depicted. Dark input droplets containing a high concentration of bromophenol blue (fluid A) are introduced into microchannel 710. Injection with clear fluid B at aperture 714 produces a ‘half-and-half’ droplet with a distinct gradient of fluid A at the lower portion of the droplet to fluid B at the top portion of the droplet (on the aperture side of the microchannel 710). Upon splitting at the fork 716 close to the injection aperture 714, outlet 1 droplets in branch 717 contain mostly fluid B as indicated by the low absorption of the droplet, compared to droplets in branch 718 (outlet 2) which retain some appreciable fraction of absorptivity due to a higher concentration of bromophenol blue. This asymmetry after splitting close to the injection aperture demonstrates slow diffusive, advective and convective mixing prior to splitting.

### 4.5 Magnetic Particle Migration

The magnetic particle migration process (also called margination process herein because particles are intended to migrate to one margin of the droplet) is highly dependent on a variety of factors such as flow rate (droplet velocity), injection rate, the distance D between injection and splitting, magnetic particle type (paramagnetic, superparamagnetic etc.) and microparticle size (nanoparticles or microparticles). Effective bead migration can be seen demonstrated in FIG. 8A through FIG. 8F.

FIG. 8A through FIG. 8F are time lapse images that illustrates example nanoparticle migration and separation in experimental apparatus in operation for high-throughput exchange of fluids in droplets containing magnetic nanoparticles, according to an embodiment. FIG. 8A is labeled to point out a picoinjector including supply channel 812, aperture 814 and electrodes 821 and 822. FIG. 8A also illustrates microchannel 810 that is wider immediately downstream of aperture 814 relative to the width immediately upstream of aperture 814, thus R3>0. Also depicted is magnet 832, fork 816, and output 1 branch 817 for washed droplets and output 2 branch 818 for waste droplets, the two branches of equal width so R1=1. A particular droplet 893 is outlined. FIG. 8B through 8F, show the same apparatus at different times labeled in bold white numerals in milliseconds (ms) after the image of FIG. 8A. The same droplet 893 is outlined in each frame as it moves through the device.

In this experiment, superparamagnetic microparticles were used (˜0.6 micron diameter) as beads. The beads can be seen in a uniformly distributed state prior to the injection channel and prior to reaching the magnetic field from the stationary rare earth magnet 832. The droplet is injected with wash fluid, and as the droplet approaches closer to the magnet, the microparticles migrate to the top half of the droplet. This partitioning immediately precedes splitting of the droplet at a fork leading to two outlet channels, outlet 1 (branch 817) and outlet 2 (branch 818). Frame 4 (FIG. 8D) shows the majority of magnetic microparticles in the upper half of the droplet indicating proper conditions for almost complete recovery of microparticles post splitting. Furthermore the white arrow, in each of FIG. 8D through FIG. 8F, points to a cluster of beads that have aggregated. The cluster is properly re-located within the droplet 893 to be included in the washed channel 817 after splitting at the fork 816.

### 4.6 Dual Stage Washing

The described device can be implemented in serial so as to obtain higher fluid exchange efficiency. This involves the duplication of integral components such as the picoinjector, magnet, and fork. The fluid resistance should be matched between the first junction outlet 2 (waste droplet branch) and the remaining second stage of the described device, as described above.

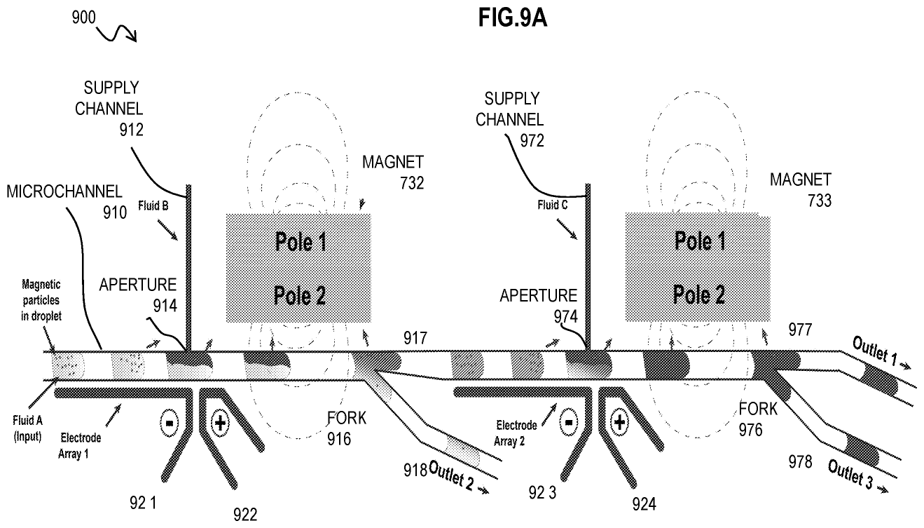

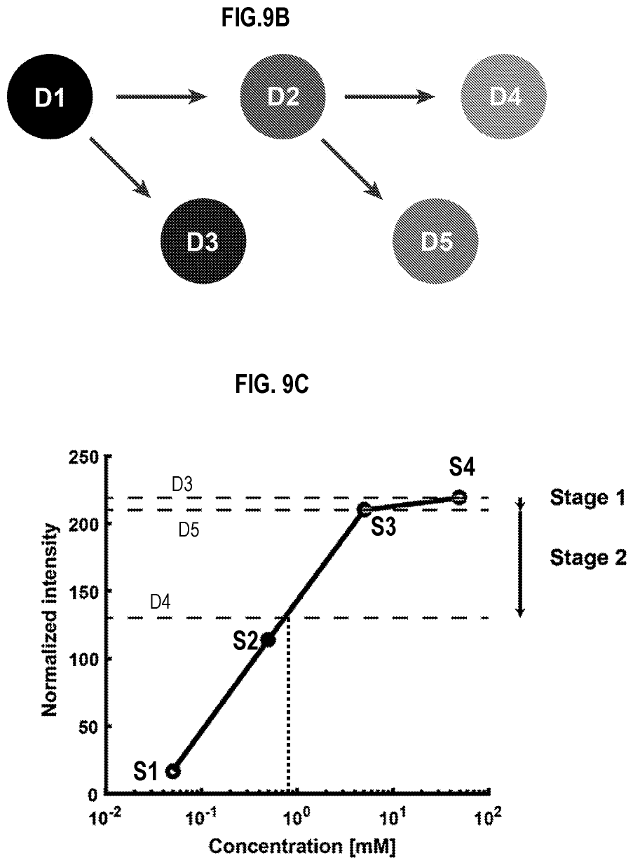

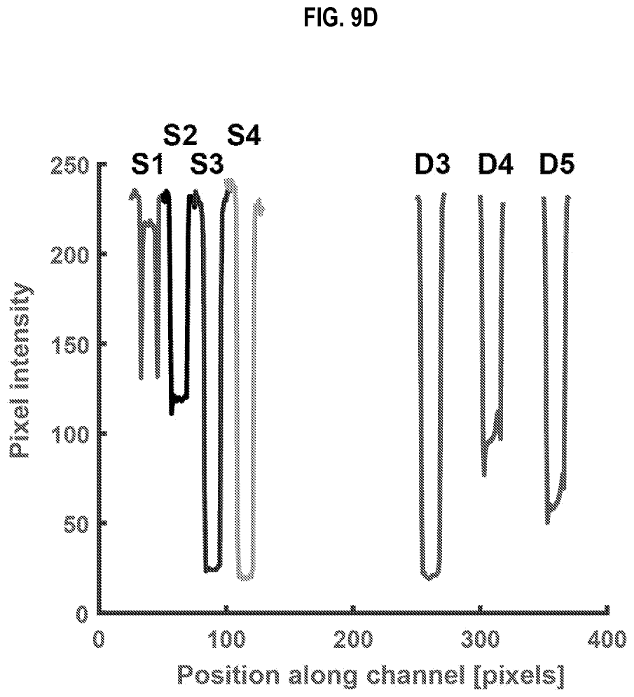

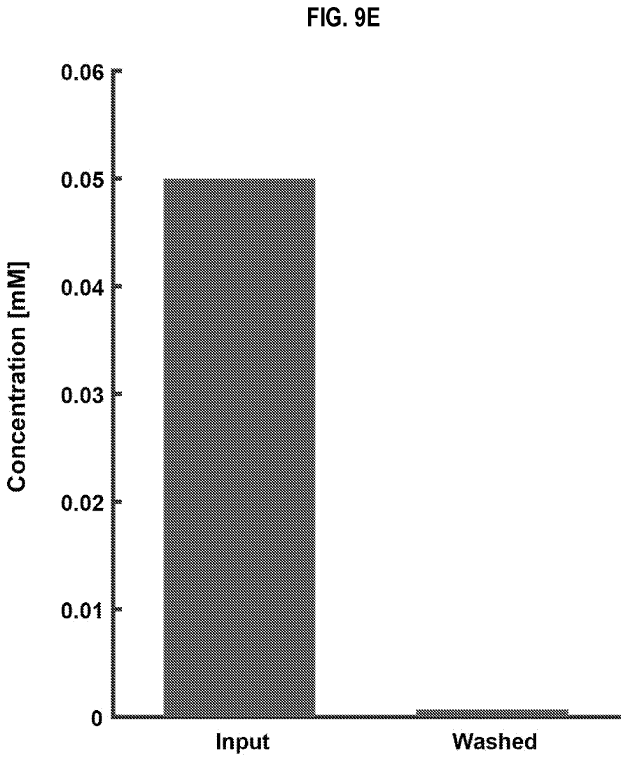

FIG. 9A is a block diagram that illustrates an example dual stage fluid exchange in experimental apparatus 900 in operation for high-throughput, continuous exchange of fluids in droplets, according to an embodiment. The microfluidic apparatus 900 includes, in downstream order, microchannel 910, magnet 732, fork 916, washed branch 917 (stage 1 outlet 1), waste branch 918 (stage 1 outlet 2), magnet 733, fork 976, twice washed branch 977 (stage 2 outlet 1), once-washed waste branch 978 (stage 2 outlet 2). The apparatus 900 also includes, upstream of magnet 732, a first picoinjector that includes supply channel 912, aperture 914 and electrodes 921 and 922. The apparatus 900 also includes, downstream of fork 916 and upstream of magnet 733, a second picoinjector that includes supply channel 972, aperture 974 and electrodes 923 and 924. Although shown for illustrating operation, the apparatus 900 does not include spacer fluid, droplets, magnetic particles, fluid A, fluid B, or fluid C. Fluid C is the wash fluid for the second stage. Typically, fluid C is the same wash buffer as fluid B.

FIG. 9B is a diagram that illustrates dual stage fluid exchange implemented in the apparatus of FIG. 9A, according to an embodiment. FIG. 9B shows a schematic of the dilution process for an original droplet of bromophenol blue dye as it transits the dual stage module. D1 is the input droplet and is darkest. D2 is the washed droplet at outlet 1 of the first stage of the module and is lighter. D3 is the waste droplet at outlet 2 of the first stage of the module and is nearly as dark as D1. Washed droplet D2 is subsequently the input for the second stage of the module. D4 is the re-washed droplet at outlet 1 of the second stage of the module and is the lightest of all. D5 is the once washed waste droplet at outlet 2 of the second stage of the module and is about the same intensity as D2.

Dual stage washing and washing efficiency or fluid exchange efficiency was demonstrated in this experiment using a bromophenol blue absorbance measurement. The absorbance of a known set of concentrations of bromophenol blue (BPB) dye in uniformly sized droplets was used to generate a calibration curve. Known input dye concentrations were used in the described device and output droplet absorbance measurements were compared to the standard curve for an estimation of washing efficiency or fluid replacement efficiency. FIG. 9C is a graph that illustrates a calibration curve of concentration of dye in known reference droplets, according to an embodiment. The horizontal axis indicates dye concentration in milliMolar (mM, 1 mM=10⁻³ Molar) on a logarithmic scale. The vertical axis indicates normalized intensity in arbitrary units Normalized intensity means an absolute value of a difference in intensity measured for the interior of a droplet relative to the background. In this plot the normalized intensity is high because the difference is high when the dye concentration is high in the droplet compared to the spacer fluid without dye. The solid circles connected by the solid line indicates the dye concentrations used to develop four standard intensities S1 through S4 at dye concentrations of approximately 50 mM, 5 mM, 0.5 mM, and 0.05 mM, respectively, yielding normalized intensities of about 220, 210, 120 and 20, respectively. This calibration curve can be used to map the intensity observed at each washing output described below and indicated by the horizontal dashed lines, to a corresponding dye concentration. The change in dye concentrations then gives the washing efficiency.

FIG. 9D is a graph that illustrates concentration of dye in output droplets from stage 1 and stage 2 of the equipment of FIG. 9A compared to the concentration of dye in known reference droplets, according to an embodiment. The horizontal axis indicates pixel position along an image of a device, such as the device diagrammed in FIG. 9A. The vertical axis indicates non normalized pixel intensity, so low values indicate high concentrations of dye (the reverse of the vertical axis in FIG. 9C). Upstream of the first picoinjector, four droplets are in the microchannel 910 from the four standards S1, through S4 in order. The pixels in each droplet show the intensity from the calibration curve at 220, 210, 120 and 20, respectively—indicating ever higher concentrations of dye. The pixels in the spacer fluid between each droplet show high intensity because there is no dye in the spacer fluid. By measuring the absorbance of droplets D3, D5 and D4 at about 20, 100 and 60, respectively, we can determine the dye concentrations from the dashed lines in FIG. 9C and thus estimate the overall fluid exchange efficiency via these absorbance measurements.

FIG. 9E is a graph that illustrates comparison of concentration of dye in input droplets to stage 1 compared to concentration of dye in output droplets from stage 2 of the equipment of FIG. 9A, according to an embodiment. The input concentration (D1) and washed (D4) concentration are plotted. The vertical axis indicates dye concentration in Molar and the horizontal axis indicates whether washed or input. Two stage fluid exchange reduced the concentration of BPB dye from 50 mM to approximately 0.5 mM as measured via absorbance and compared to the standard calibration curve. This represents a 98.6% reduction in concentration.

### 4.6 Bead Migration Along Channel Distance, D

The distance D of the channel immediately following injection affects whether there is sufficient bead margination and retention during operation. The distance D that should be used to configure the microfluidic device depends on factors such as flow rate, bead size, type of fluids, and injection volume (pressure and voltage at picoinjector). This can be determined by experimentation for any particular washing operation.

For example, an experiment was conducted to measure the trajectory of 22 micron paramagnetic microparticles within a droplet under influence from a N52 rare earth permanent magnet situated immediately adjacent to the post injection section of the microchannel Tracking the positions for a variety of droplets of the magnetic microparticles gives a picture of how the microparticles are perturbed after injection and how much distance is required for them to return to a desired position on the wash fluid (fluid B) side (aperture side) of the split line of the fork, given the boundary constraints of the droplet and the microchannel walls.

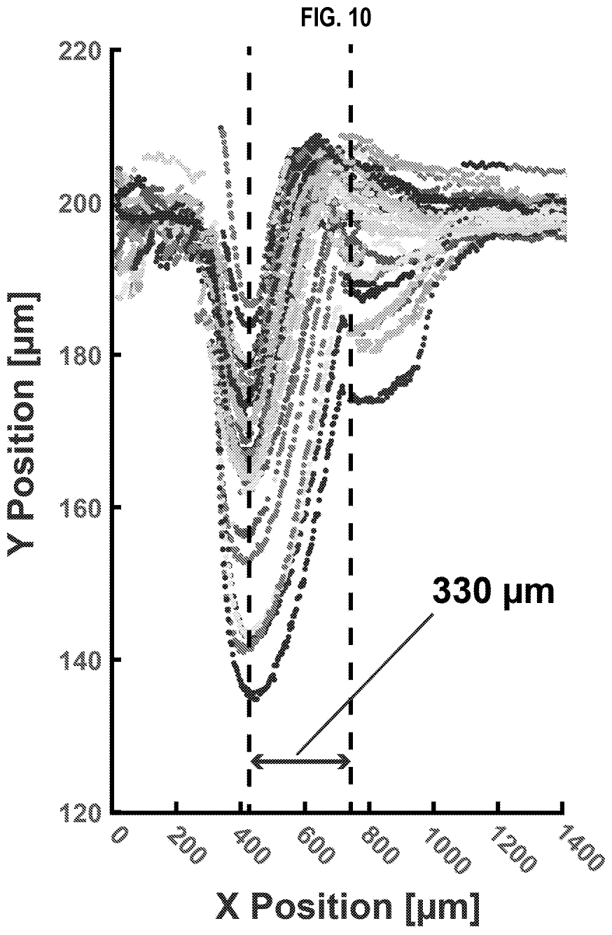

FIG. 10 is a graph that illustrates example migration of magnetic nanoparticles within a droplet as a droplet streams along the microchannel of FIG. 2A, according to an embodiment. The horizontal axis indicates positon along the device in microns. The vertical axis indicates position across the microchannel 210 in microns, with higher values closer to the microchannel wall with the aperture 114 of the picoinjector. Magnetic microparticle tracking was performed using high speed video analysis. The trajectories of the microparticles can be seen to originate from the aperture side of the droplet (higher Y position) as they were pre-oriented using a permanent magnet 231 just upstream of the injection aperture. After injection, the position of the beads is deflected by approximately 15 microns to 60 microns to lower values of Y. Under the influence of the magnetic field from the permanent magnet 232 just downstream of the injection channel, for these flow rates, microparticle type, microparticle size, it takes approximately 330 microns for the paramagnetic microparticles to return to their original distribution. Thus an optimal distance between the injection channel and the splitting junction should be slightly more than this value to ensure enough time for proper microparticle margination to the larger Y coordinate portion of the droplet. The flow rates for the illustrated experimental embodiment was about 150 μl per hour for the aqueous drops and about 400 μl per hour for the spacer oil.

### 5. COMPUTATIONAL HARDWARE OVERVIEW

FIG. 11 is a block diagram that illustrates a computer system 1100 upon which an embodiment of the invention may be implemented. Computer system 1100 includes a communication mechanism such as a bus 1110 for passing information between other internal and external components of the computer system 1100. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit)). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 1100, or a portion thereof, constitutes a means for performing one or more steps of one or more methods described herein.

A sequence of binary digits constitutes digital data that is used to represent a number or code for a character. A bus 1110 includes many parallel conductors of information so that information is transferred quickly among devices coupled to the bus 1110. One or more processors 1102 for processing information are coupled with the bus 1110. A processor 1102 performs a set of operations on information. The set of operations include bringing information in from the bus 1110 and placing information on the bus 1110. The set of operations also typically include comparing two or more units of information, shifting positions of units of information, and combining two or more units of information, such as by addition or multiplication. A sequence of operations to be executed by the processor 1102 constitutes computer instructions.

Computer system 1100 also includes a memory 1104 coupled to bus 1110. The memory 1104, such as a random access memory (RAM) or other dynamic storage device, stores information including computer instructions. Dynamic memory allows information stored therein to be changed by the computer system 1100. RAM allows a unit of information stored at a location called a memory address to be stored and retrieved independently of information at neighboring addresses. The memory 1104 is also used by the processor 1102 to store temporary values during execution of computer instructions. The computer system 1100 also includes a read only memory (ROM) 1106 or other static storage device coupled to the bus 1110 for storing static information, including instructions, that is not changed by the computer system 1100. Also coupled to bus 1110 is a non-volatile (persistent) storage device 1108, such as a magnetic disk or optical disk, for storing information, including instructions, that persists even when the computer system 1100 is turned off or otherwise loses power.

Information, including instructions, is provided to the bus 1110 for use by the processor from an external input device 1112, such as a keyboard containing alphanumeric keys operated by a human user, or a sensor. A sensor detects conditions in its vicinity and transforms those detections into signals compatible with the signals used to represent information in computer system 1100. Other external devices coupled to bus 1110, used primarily for interacting with humans, include a display device 1114, such as a cathode ray tube (CRT) or a liquid crystal display (LCD), for presenting images, and a pointing device 1116, such as a mouse or a trackball or cursor direction keys, for controlling a position of a small cursor image presented on the display 1114 and issuing commands associated with graphical elements presented on the display 1114.

In the illustrated embodiment, special purpose hardware, such as an application specific integrated circuit (IC) 1120, is coupled to bus 1110. The special purpose hardware is configured to perform operations not performed by processor 1102 quickly enough for special purposes. Examples of application specific ICs include graphics accelerator cards for generating images for display 1114, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1100 also includes one or more instances of a communications interface 1170 coupled to bus 1110. Communication interface 1170 provides a two-way communication coupling to a variety of external devices that operate with their own processors, such as printers, scanners and external disks. In general the coupling is with a network link 1178 that is connected to a local network 1180 to which a variety of external devices with their own processors are connected. For example, communication interface 1170 may be a parallel port or a serial port or a universal serial bus (USB) port on a personal computer. In some embodiments, communications interface 1170 is an integrated services digital network (ISDN) card or a digital subscriber line (DSL) card or a telephone modem that provides an information communication connection to a corresponding type of telephone line. In some embodiments, a communication interface 1170 is a cable modem that converts signals on bus 1110 into signals for a communication connection over a coaxial cable or into optical signals for a communication connection over a fiber optic cable. As another example, communications interface 1170 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 1170 sends and receives electrical, acoustic or electromagnetic signals, including infrared and optical signals, that carry information streams, such as digital data.

The term computer-readable medium is used herein to refer to any medium that participates in providing information to processor 1102, including instructions for execution. Such a medium may take many forms, including, but not limited to, non-volatile media, volatile media and transmission media. Non-volatile media include, for example, optical or magnetic disks, such as storage device 1108. Volatile media include, for example, dynamic memory 1104. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 1102, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 1102, except for carrier waves and other signals.

Logic encoded in one or more tangible media includes one or both of processor instructions on a computer-readable storage media and special purpose hardware, such as ASIC 1120.

Network link 1178 typically provides information communication through one or more networks to other devices that use or process the information. For example, network link 1178 may provide a connection through local network 1180 to a host computer 1182 or to equipment 1184 operated by an Internet Service Provider (ISP). ISP equipment 1184 in turn provides data communication services through the public, world-wide packet-switching communication network of networks now commonly referred to as the Internet 1190. A computer called a server 1192 connected to the Internet provides a service in response to information received over the Internet. For example, server 1192 provides information representing video data for presentation at display 1114.

The invention is related to the use of computer system 1100 for implementing the techniques described herein. According to one embodiment of the invention, those techniques are performed by computer system 1100 in response to processor 1102 executing one or more sequences of one or more instructions contained in memory 1104. Such instructions, also called software and program code, may be read into memory 1104 from another computer-readable medium such as storage device 1108. Execution of the sequences of instructions contained in memory 1104 causes processor 1102 to perform the method steps described herein. In alternative embodiments, hardware, such as application specific integrated circuit 1120, may be used in place of or in combination with software to implement the invention. Thus, embodiments of the invention are not limited to any specific combination of hardware and software.

The signals transmitted over network link 1178 and other networks through communications interface 1170, carry information to and from computer system 1100. Computer system 1100 can send and receive information, including program code, through the networks 1180, 1190 among others, through network link 1178 and communications interface 1170. In an example using the Internet 1190, a server 1192 transmits program code for a particular application, requested by a message sent from computer 1100, through Internet 1190, ISP equipment 1184, local network 1180 and communications interface 1170. The received code may be executed by processor 1102 as it is received, or may be stored in storage device 1108 or other non-volatile storage for later execution, or both. In this manner, computer system 1100 may obtain application program code in the form of a signal on a carrier wave.

Various forms of computer readable media may be involved in carrying one or more sequence of instructions or data or both to processor 1102 for execution. For example, instructions and data may initially be carried on a magnetic disk of a remote computer such as host 1182. The remote computer loads the instructions and data into its dynamic memory and sends the instructions and data over a telephone line using a modem. A modem local to the computer system 1100 receives the instructions and data on a telephone line and uses an infra-red transmitter to convert the instructions and data to a signal on an infra-red a carrier wave serving as the network link 1178. An infrared detector serving as communications interface 1170 receives the instructions and data carried in the infrared signal and places information representing the instructions and data onto bus 1110. Bus 1110 carries the information to memory 1104 from which processor 1102 retrieves and executes the instructions using some of the data sent with the instructions. The instructions and data received in memory 1104 may optionally be stored on storage device 1108, either before or after execution by the processor 1102.

FIG. 12 illustrates a chip set 1200 upon which an embodiment of the invention may be implemented. Chip set 1200 is programmed to perform one or more steps of a method described herein and includes, for instance, the processor and memory components described with respect to FIG. 11 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1200, or a portion thereof, constitutes a means for performing one or more steps of a method described herein.

In one embodiment, the chip set 1200 includes a communication mechanism such as a bus 1201 for passing information among the components of the chip set 1200. A processor 1203 has connectivity to the bus 1201 to execute instructions and process information stored in, for example, a memory 1205. The processor 1203 may include one or more processing cores with each core configured to perform independently. A multi-core processor enables multiprocessing within a single physical package. Examples of a multi-core processor include two, four, eight, or greater numbers of processing cores. Alternatively or in addition, the processor 1203 may include one or more microprocessors configured in tandem via the bus 1201 to enable independent execution of instructions, pipelining, and multithreading. The processor 1203 may also be accompanied with one or more specialized components to perform certain processing functions and tasks such as one or more digital signal processors (DSP) 1207, or one or more application-specific integrated circuits (ASIC) 1209. A DSP 1207 typically is configured to process real-world signals (e.g., sound) in real time independently of the processor 1203. Similarly, an ASIC 1209 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1203 and accompanying components have connectivity to the memory 1205 via the bus 1201. The memory 1205 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1205 also stores the data associated with or generated by the execution of one or more steps of the methods described herein.

### 6. ALTERATIONS, IMPROVEMENTS AND MODIFICATIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 7. REFERENCES

The entire contents of each of the following references is hereby incorporated as if wholly recited herein, except for terminology that is inconsistent with the terminology used herein.
Hun Lee, Linfeng Xu and Kwang Oh, “Droplet-based microfluidic washing module for magnetic particle-based assays,” Biomicrofluidics, vol. 8, #044113, 2014
Ramakrishna Sista, Allen Eckhardt, Vijay Srinivasan, Michael Pollack, Srinivas Palanki and Vamsee Pamula, “Heterogeneous immunoassays using magnetic beads on a digital microfluidic platform,” Lab on a Chip, vol. 8, pp 2188-2196, 14 Oct. 2008
Adam R. Abate, Tony Hung, Pascaline Mary, Jeremy J. Agresti, and David A. Weitz, “High-throughput injection with microfluidics using picoinjectors,” Proc Natl Acad Sci USA. 2010 Nov. 9; 107(45): 19163-19166, Published online 2010 Oct. 20.
Kwang Oh, Kangsun Lee, Byungwook Ahn and Edward Furlani, “Design of pressure-driven microfluidic networks using electric circuit analogy, Lab on a Chip, Issue 3, 2012.

1. An apparatus comprising:
a substrate having formed thereon
a microchannel configured to pass a continuous stream of a plurality of droplets comprising a first fluid with a plurality of magnetic particles, the plurality of droplets separated in the stream by a spacer fluid,
a plurality of microchannel branches comprising a first microchannel branch and a different second microchannel branch, and
a fork comprising a junction between the microchannel upstream of the fork and the plurality of microchannel branches downstream of the fork;
a picoinjector disposed along the microchannel, the picoinjector comprising a supply channel formed in the substrate and connected to the microchannel by an aperture on a first side of the microchannel and a pair of electrodes on an opposite side of the microchannel, wherein the aperture is a distance D upstream of the fork, the picoinjector configured to inject through the aperture a volume of a second fluid into a first droplet of the plurality of droplets in the stream when the pair of electrodes carry at least a certain voltage difference; and
a first magnet disposed adjacent to the microchannel between the picoinjector and the fork and configured to introduce a magnetic field into the microchannel between the picoinjector and the fork to move magnetic particles in the first droplet toward the first side of the microchannel.

2. An apparatus as recited in claim 1, further comprising a different second magnet disposed adjacent to the microchannel upstream of the aperture of the picoinjector and configured to introduce a magnetic field into the microchannel to move magnetic particles in the first droplet toward the first side of the microchannel.

3. An apparatus as recited in claim 1, wherein:
the first microchannel branch is spaced relative to the second microchannel branch in a direction parallel to a direction in which the magnetic particles are forced by the field of the first magnet;
a cross sectional area of the first microchannel branch is R1 times a cross sectional area of the second microchannel branch;
the picoinjector is configured to inject a volume that is R2 times a volume of the first droplet upstream of the aperture; and
R2≈R1.

4. An apparatus as recited in claim 3, wherein R1 is different from 1.

5. An apparatus as recited in claim 3, wherein R1>1.

6. An apparatus as recited in claim 1, wherein:
a distance D from the aperture to the fork divided by a migration time (T_{M}) defines a speed of the stream during operation; and
T_{M} is short compared to a time for the second fluid to mix with the first fluid in the droplet and long compared to a time for the magnetic particles to move in the magnetic field of the first magnet at least a tenth of the width of the microchannel between the electrodes and the fork.

7. An apparatus as recited in claim 1, wherein D is a value within a range from about 10 microns to about 1000 microns.

8. An apparatus as recited in claim 1, wherein the microchannel has a first cross sectional area in the vicinity of the aperture that is less than a second cross sectional area of the microchannel between the electrodes and the fork.

9. An apparatus as recited in claim 1, wherein the microchannel has a first cross sectional area in the vicinity of the aperture that is less than a second cross sectional area of the microchannel upstream of the aperture.

10. An apparatus as recited in claim 1, wherein the microchannel has a first cross sectional area immediately upstream of the aperture that is less than a second cross sectional area of the microchannel immediately downstream of the aperture.

11. An apparatus as recited in claim 10, wherein:
the second cross sectional area is (1+R3) times the first cross sectional area;
the picoinjector is configured to inject a volume that is R2 times a volume of the first droplet upstream of the aperture; and
R2 is based on R3.

12. An apparatus as recited in claim 1, wherein the first magnet is an electromagnet.

13. An apparatus as recited in claim 1, wherein the first magnet is a permanent magnet.

14. An apparatus as recited in claim 13, wherein the first magnet is a rare earth Neodymium magnet.

15. A method comprising:
causing a stream of a plurality of droplets separated by a spacer fluid, wherein each droplet of the plurality of droplets comprises a first fluid with a plurality of magnetic particles, to flow through a device comprising
a microchannel,
a plurality of microchannel branches comprising a first microchannel branch and a different second microchannel branch,
a fork comprising a junction between the microchannel upstream of the fork and the plurality of microchannel branches downstream of the fork,
a picoinjector disposed along the microchannel, the picoinjector comprising a supply channel connected to the microchannel by an aperture on a first side of the microchannel and a pair of electrodes on an opposite side of the microchannel, wherein the aperture is a distance D upstream of the fork and
a first magnet disposed adjacent to the microchannel between the picoinjector and the fork;
supplying a second fluid to the supply channel;
applying a voltage difference to the pair of electrodes when a first droplet of the plurality of droplets is in contact with the second fluid at the aperture to inject through the aperture a volume of the second fluid into the first droplet;
introducing from the first magnet a magnetic field into the microchannel between the picoinjector and the fork to move magnetic particles in the first droplet toward the first side of the microchannel; and
collecting an output droplet from the first microchannel branch.

16. A method as recited in claim 15, wherein the first microchannel branch is spaced relative to the second microchannel branch in a direction parallel to a direction in which the magnetic particles are forced by the field of the first magnet, wherein the output droplet is divided from the first droplet at the fork, whereby the output droplet comprises the second fluid and at least some of the plurality of magnetic particles.

17. A method as recited in claim 15, wherein each magnetic particle of the plurality of magnetic particles is a paramagnetic particle or a superparamagnetic particle.

18. A method as recited in claim 15, wherein each magnetic particle of the plurality of magnetic particles is connected to a species of interest to be washed with the second fluid.

19. A method as recited in claim 16, wherein the output droplet comprises the second fluid and most of the plurality of magnetic particles.

20. A method as recited in claim 19, wherein the output droplet comprises more of the second fluid than the first fluid, whereby the magnetic particles have been washed by the second fluid.

21. A method as recited in claim 15, wherein:
the device further comprising a different second magnet disposed adjacent to the microchannel upstream of the aperture of the picoinjector; and
the method further includes introducing from the second magnet a magnetic field into the microchannel upstream of the aperture of the picoinjector to move magnetic particles in the first droplet toward the first side of the microchannel before injecting the volume of the second fluid into the first droplet.

22. A method as recited in claim 15, wherein:
a cross sectional area of the first microchannel branch is R1 times a cross sectional area of the second microchannel branch;
the volume of the second fluid injected into the first droplet is R2 times a volume of the first droplet upstream of the aperture; and
R2≈R1.

23. A method as recited in claim 22, wherein R1 is different from 1.

24. A method as recited in claim 22, wherein R1>1.

25. A method as recited in claim 15, wherein:
causing the stream to flow through the device further comprises causing the stream to flow at a speed given by a distance D from the aperture to the fork divided by a migration time (T_{M}); and
T_{M} is short compared to a time for the second fluid to mix with the first fluid in the first droplet and long compared to a time for the magnetic particles to move in the magnetic field of the first magnet at least a tenth of a width of the microchannel between the electrodes and the fork.

26. A system comprising:
the apparatus of claim 1;
a pressure actuator;
a processor; and
a computer-readable medium including one or more sequences of instructions, the computer-readable medium and the one or more sequences of instructions configured to, with the processor, cause the system perform at least the following:
operate the pressure actuator to cause a stream of a plurality of droplets separated by a spacer fluid, wherein each droplet of the plurality of droplets comprises a first fluid with a plurality of magnetic particles, to flow through the apparatus;
operate the pressure actuator to supply a second fluid to the supply channel;
apply a voltage difference to the pair of electrodes when a first droplet of the plurality of droplets is in contact with the second fluid at the aperture to inject through the aperture a volume of the second fluid into the first droplet;
introduce from the first magnet a magnetic field into the microchannel between the picoinjector and the fork to move magnetic particles in the first droplet toward the first side of the microchannel; and
collect an output droplet from the first microchannel branch that is spaced relative to the second microchannel branch in a direction parallel to a direction in which the magnetic particles are forced by the field of the first magnet, wherein the output droplet is divided from the first droplet at the fork, whereby the output droplet comprises the second fluid and at least some of the plurality of magnetic particles.

27. An apparatus comprising:
a processor; and
a computer-readable medium including one or more sequences of instructions, the computer-readable medium and the one or more sequences of instructions configured to, with the processor, cause a system perform at least the following:
operate a pressure actuator to cause a stream of a plurality of droplets separated by a spacer fluid to flow in a microchannel, wherein each droplet of the plurality of droplets comprises a first fluid with a plurality of magnetic particles, to flow through the apparatus;
operate the pressure actuator to supply a second fluid to a supply channel of a picoinjector;
apply a voltage difference to a pair of electrodes in the picoinjector when a first droplet of the plurality of droplets is in contact with the second fluid at an aperture of the picoinjector on a first side of the microchannel to inject through the aperture a volume of the second fluid into the first droplet;
introduce from a first magnet a magnetic field into the microchannel between the picoinjector and a fork to move magnetic particles in the first droplet toward the first side of the microchannel; and
collect an output droplet from a first microchannel branch downstream of the fork.

28. A non-transitory computer-readable medium including one or more sequences of instructions, the computer-readable medium and the one or more sequences of instructions configured to cause a system to perform at least the following:
operate a pressure actuator to cause a stream of a plurality of droplets separated by a spacer fluid to flow in a microchannel, wherein each droplet of the plurality of droplets comprises a first fluid with a plurality of magnetic particles, to flow through the apparatus;
operate the pressure actuator to supply a second fluid to a supply channel of a picoinjector;
apply a voltage difference to a pair of electrodes in the picoinjector when a first droplet of the plurality of droplets is in contact with the second fluid at an aperture of the picoinjector on a first side of the microchannel to inject through the aperture a volume of the second fluid into the first droplet;
introduce from a first magnet a magnetic field into the microchannel between the picoinjector and a fork to move magnetic particles in the first droplet toward the first side of the microchannel; and
collect an output droplet from a first microchannel branch downstream of the fork.

29. An output droplet in a spacer fluid derived from an input droplet in the spacer fluid, wherein:
the input droplet comprises a first fluid with a plurality of magnetic particles; and
the output droplet comprises a different second fluid and the plurality of magnetic particles.

30. An output droplet as recited in claim 29, wherein the output droplet is formed by:
applying a voltage difference to a pair of electrodes when the input droplet is in contact with a supply of the second fluid at an aperture on a first side of a microchannel to inject through the aperture a volume of the second fluid into the input droplet to form a transition droplet;
introducing a magnetic field to move the plurality of magnetic particles in the transition droplet toward the first side of the microchannel; and
splitting the transition droplet at a fork in the microchannel into the output droplet in a first branch microchannel downstream of the fork and into a waste droplet in a different second branch microchannel downstream of the fork.
